(19) United States
(12) Patent Application Publication
(10) Pub. No.: US 2018/0271820 A1
(43) Pub. Date: Sep. 27, 2018

(54) METHODS OF TREATING HEART FAILURE

(71) Applicant: Vijaykumar Rajasekhar, Apple Valley, CA (US)

(72) Inventor: Vijaykumar Rajasekhar, Apple Valley, CA (US)

(21) Appl. No.: 15/984,605

(22) Filed: May 21, 2018

Related U.S. Application Data
(63) Continuation-in-part of application No. 15/249,744, filed on Aug. 29, 2016.
Application No. 15/249,744 is a continuation-in-part of application No. 15/457,908, filed on Mar. 13, 2017.
(60) Provisional application No. 62/210,738, filed on Aug. 27, 2015.
(60) Provisional application No. 62/307,474, filed on Mar. 12, 2016.

(51) Int. Cl. A61K 31/225; A61K 9/16; A61P 9/04

(52) U.S. Cl. CPC A61K 31/225; A61K 9/4891; A61P 9/04; A61K 9/16

(57) ABSTRACT

Pharmaceutical preparations and methods for treating or preventing a heart failure disease, including heart failure with preserved ejection fraction (HFPEF), with one or more dialkyl fumarates alone or in combination with one or more second agents are provided herein. In some embodiments, the pharmaceutical preparations are in the form of micro-tablets or pellets.

### CROSS-REFERENCE TO RELATED APPLICATIONS

This application is a continuation-in-part of U.S. patent application Ser. No. 15/249,744, filed on Aug. 29, 2016, which claims the benefit of U.S. Provisional Patent Application Ser. No. 62/210,738, filed on Aug. 27, 2015, both of which are herein incorporated by reference in their entireties.

This application is also a continuation-in-part of U.S. patent application Ser. No. 15/457,908, filed Mar. 13, 2017, which claims the benefit of U.S. Provisional Patent Application Ser. No. 62/307,474, filed on Mar. 12, 2016, both of which are herein incorporated by reference in their entireties.

### INCORPORATION BY REFERENCE

All publications and patent applications mentioned in this specification are herein incorporated by reference in their entireties, as if each individual publication or patent application was specifically and individually indicated to be incorporated by reference in its entirety.

### TECHNICAL FIELD

The present disclosure relates to the fields of cardiology and pharmaceutical sciences. In particular, the present disclosure relates to certain pharmaceutical preparations comprising dialkyl fumarates and administering said pharmaceutical preparations alone or in combination with one or more second agents for the treatment of a heart failure disease including, heart failure with preserved ejection fraction.

This disclosure further relates to methods and compositions for treating or preventing a heart failure disease, including heart failure with preserved ejection fraction (HFPEF), in a subject in need thereof by administering to the subject a therapeutically effective amount of a Fatty acid fumarate derivative (FAFD) alone or in combination with one or more second agents useful for treating heart failure.

### BACKGROUND

Heart failure (HF) is a major health problem in the United States (U.S.) and elsewhere. In the U.S., HF affects over 5 million people with approximately half a million new cases occurring each year. HF is the leading cause of hospitalizations in people over 65 years in age. HF has many potential causes and diverse clinical features. Symptoms of heart failure can include dyspnea during activity or at rest, cough with white sputum, rapid weight gain, swelling in ankles, legs and abdomen, dizziness, fatigue and weakness, rapid or irregular heartbeats, nausea, palpitations, and chest pains.

About half of heart failure patients have HF with preserved ejection fraction (HFPEF). In traditional HF (i.e., heart failure with reduced ejection fraction (HFREF)), the ventricle cannot contract normally. However in patients with HFPEF, the declined performance of the heart ventricle is not at the time of contraction (systole), but during the relaxation/filling phase of diastole. HFPEF patients show normal ejection fraction of blood pumped out of the ventricle, but the heart muscle does not quickly relax to allow efficient filling of blood returning from the body. Morbidity and mortality of HFPEF are similar to HFREF; however, therapies that treat HFREF are not effective in treating or preventing HFPEF. Patients with HFPEF have an ejection fraction of ≥40%, ≥45%, or ≥50% depending on which definition is chosen from the literature. On the other hand, patients with HFREF have an ejection fraction of either ≤35% or ≤40% depending on which definition and guidelines are used. For ease of simplicity, and not to be limiting in any way, HFPEF can be considered as having an ejection fraction ≥40% and HFREF can be considered as having an ejection fraction <40%.

Other names for the two primary clinical subsets of HF are diastolic heart failure (DHF) and systolic heart failure (SHF). SHF, which is also known as heart failure with reduced ejection fraction (HFREF) involves an abnormality of the heart resulting in failure of the heart to pump blood at a rate needed for metabolizing tissues at rest and/or during exertion. DHF, which is also known as heart failure with preserved ejection fraction (HFPEF), is a clinical syndrome with symptoms and signs of HF, a preserved ejection fraction, and abnormal diastolic function. The clinical manifestations of HFREF and HFPEF have distinct differences in risk factors, patient characteristics, and pathophysiology. Moreover, medications proven effective in HFREF have not been found to be effective in HFPEF. At present there are no approved treatments to reduce mortality in HFPEF.

In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in patients with HFPEF and are not approved therapies for HFPEF.

Given that there are currently no approved treatments to improve survival in HFPEF, there remains, therefore, a real urgent need for a product that can improve morbidity and mortality of patients with HFPEF.

The present disclosure addresses these needs in patients with HFPEF as well as in patients at risk of developing HFPEF, due to conditions including but not limited to hypertension, diabetes, COPD, atrial fibrillation, obesity, or ischemic heart disease.

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been shown to improve in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in subjects at risk of a cardiovascular event. Omega-3 fatty acids have also been taken as dietary supplements, as treatment of dyslipidemia, and as an anti-inflammatory agent. A higher intake of omega-3 fatty acids lower levels of circulating TNF-α and IL-6, two of the cytokines that are markedly increased during inflammation processes (Chapkin et al, Prostaglandins, Leukot Essent Fatty Acids 2009, 81, p. 187-191; Duda et al, Cardiovasc Res 2009, 84, p. 33-41). In addition, a higher intake of omega-3 fatty acids has been shown to increase levels of the well-characterized anti-inflammatory cytokine IL-10 (Bradley et al, Obesity (Silver Spring) 2008, 16, p. 938-944). A recent study (Wang et al, Molecular Pharmaceutics 2010, 7, p. 2185-2193) has demonstrated that DHA could also induce the Nrf2 and the Nrf2-target gene Heme-oxygenase 1 (HO-1). This pathway could play a significant role in suppressing LPS-mediated inflammation.

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as a free radical scavenger, chelator to heavy metals and signal transduction mediator in various inflammatory and metabolic pathways, including the NF-κB pathway (Shay, K. P. et al. Biochim. Biophys. Acta 2009, 1790, 1149-1160). Lipoic acid has been found to be useful in the treatment of a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

O'Connell et. al, WO 2013/116194 have disclosed a method for treating or limiting development of heart failure with preserved ejection fraction (HFPEF), comprising administering to a subject, having or at risk of developing HFPEF, an effective amount of a pharmaceutical composition comprising docosahexaenoic acid (DHA) or pharmaceutically acceptable salts, esters, amides, epoxides, and prodrugs thereof, to treat or limit development of HFPEF.

Fumaric acid and its ester derivatives, either the mono alkyl hydrogen fumarates or dialkyl fumarates, have been used as therapeutic agents for the treatment of psoriasis (Joshi and Strebel, WO 1999/49858; U.S. Pat. No. 6,277,882; Mrowietz and Asadullah, Trends Mol Med 2005, 111(1), 43-48; and Yazdi and Mrowietz, Clinics Dermatology 2008, 26, 522-526); asthma and chronic obstructive pulmonary diseases (Joshi et al, WO 2005/023241 and US 2007/0027076); cardiac insufficiency including left ventricular insufficiency, myocardial infarction and angina pectoris (Joshi et al, WO 2005/023241; Joshi et al, US 2007/0027076); mitochondrial and neurodegenerative diseases such as Parkinson's disease, Alzheimer's disease, Huntington's disease, retinopathia pigmentosa and mitochondrial encephalomyopathy (Joshi and Strebel, WO 2002/055063, US 2006/0205659, U.S. Pat. No. 6,509,376, U.S. Pat. No. 6,858,750, and U.S. Pat. No. 7,157,423); transplantation (Joshi and Strebel, WO 2002/055063, US2006/0205659, U.S. Pat. No. 6,359,003, U.S. Pat. No. 6,509,376, and U.S. Pat. No. 7,157,423; and Lehmann et al, Arch Dermatol Res 2002, 294, 399-404); autoimmune diseases (Joshi and Strebel, WO 2002/055063, U.S. Pat. No. 6,509,376, U.S. Pat. No. 7,157,423, and US 2006/0205659) including multiple sclerosis (MS) (Joshi and Strebel, WO 1998/52549 and U.S. Pat. No. 6,436,992; Went and Lieberburg, US 2008/0089896; Schimrigk et al, Eur J Neurology 2006, 13, 604-610; and Schilling et al, Clin Experimental Immunology 2006, 145, 101-107); neurological disorders characterized by extensive demyelination and/or axonal loss including secondary progressive multiple sclerosis and Devic's disease (Gold, WO 2008/096271); ischemia and reperfusion injury (Joshi et al., US 2007/0027076); AGE-induced genome damage (Heidland, WO 2005/027899); inflammatory bowel diseases such as Crohn's disease and ulcerative colitis; arthritis; others conditions (Nilsson et al., WO 2006/037342 and Nilsson and Muller, WO 2007/042034); and an autoimmune and Th1-mediated skin disease (Altmeyer et al, J. of the American Academy of Dermatology 1994, 30, p. 977-981).

In clinical studies with psoriasis patients that have been administered with fumarates, a reduction of peripheral CD4+ and CD8+-T lymphocytes has been observed. These agents have been reported to inhibit LPS-induced NF-κB activation in dendritic cells and endothelial cells in vitro (Loewe et al., J. Immunol. 2004, 168, 4781-4787; Litjens et al., Eur. J. Immunol. 2004, 34, 565-575). Dialkyl and monoalkyl fumarates have also demonstrated oral efficacy in the chronic experimental autoimmune encephalomyelitis (EAE) mouse model for multiple sclerosis (MS). In this particular model, C57BL/6 mice were challenged with the immunopeptide MOG 35-55 in order to induce disabilities that were equivalent to those exhibited by MS patients. Oral treatment with either dialkyl or monoalkyl fumarate resulted in a significant improvement in the disability score. The anti-inflammatory cytokine IL-10 was particularly elevated in the blood among the animals treated with either dialkyl or monoalkyl fumarate. Furthermore, histological analysis of the spinal cord of animals treated with either dialkyl or monoalkyl fumarate showed a strongly reduced macrophage inflammation (Schilling et al., Clinical and Experimental Immunology 2006, 145, 101-107). Dialkyl and monoalkyl fumarate esters have also been used in a number of reported studies with patients exhibiting the relapsing-remitting form of multiple sclerosis. Patients treated with 720 mg of fumarate esters daily for 70 weeks exhibited a significant reduction in inflammatory brain lesions, as noted by the reduction of new gadolinium-enhancing (Gd+) lesions in various MRI taken during the course of the treatment (Schimrigk et al., Eur. J. Neurology 2006, 13, 604-610). More recently, fumarates have been shown to activate Nrf2, a transcription factor that is responsible for the induction of a number of important antioxidants and detoxification enzymes that protect mammalian cells against reactive oxygen/nitrogen species and electrophiles (Lukashev, M. E. “Nrf2 screening assays and related methods and compositions” WO 08097596 A2; Wilms et al, Journal of Neuroinflammation 2010, 7:30).

Chronic oxidative stress and inflammation have now been linked to the development and progression of a number of debilitating diseases beyond multiple sclerosis. Activation of the Nrf2 pathway in order to resolve this chronic oxidative stress and inflammation appears to be a particularly promising new therapeutic target (For a review see Gozzelino, R. et al Annu. Rev. Pharmacol. Toxicol. 2010, 50, p. 323-54). For instance, small molecule activators of Nrf2 have now been shown to be effective in the cisplatin-induced nephrotoxicity mouse model (Aleksunes et al, J. Pharmacology & Experimental Therapeutics 2010, 335, p. 2-12), the transgenic Tg19959 mouse model of Alzheimer's disease (Dumont et al, J. Neurochem. 2009, 109, p. 502-12), the mouse model for COPD (Sussan, T. E. et al Proc. Natl. Acad. Sci. USA 2009, 106, p. 250-5), and the murine 4T1 breast tumor model (Ling, X. et al Cancer Res. 2007, 67, p. 4210-8).

FUMADERM®, an enteric coated tablet containing a salt mixture of monoethyl fumarate and dimethyl fumarate (DMF) which is rapidly hydrolyzed to monomethyl fumarate, regarded as the main bioactive metabolite, was approved in Germany in 1994 for the treatment of psoriasis. FUMADERM® is dosed three times daily (TID) with 1-2 grams/day administered for the treatment of psoriasis. FUMADERM® exhibits a high degree of interpatient variability with respect to drug absorption and food strongly reduces bioavailability. Absorption is thought to occur in the small intestine with peak levels achieved 5-6 hours after oral administration. Significant side effects occur in 70-90% of patients (Brewer and Rogers, Clin Expt'l Dermatology 2007, 32, 246-49; and Hoefnagel et al., Br J Dermatology 2003, 149, 363-369). Side effects of current FAE therapy include gastrointestinal upset including nausea, vomiting, diarrhea and/or transient flushing of the skin. Also DMF exhibits poor aqueous solubility.

Fumaric acid derivatives ((Joshi and Strebel, WO 2002/055063, US 2006/0205659, and U.S. Pat. No. 7,157,423 (amide compounds and protein-fumarate conjugates); Joshi et al, WO 2002/055066 and Joshi and Strebel, U.S. Pat. No. 6,355,676 (mono and dialkyl esters); Joshi and Strebel, WO 2003/087174 (carbocyclic and oxacarbocylic compounds); Joshi et al, WO 2006/122652 (thiosuccinates); Joshi et al, US 2008/0233185 (dialkyl and diaryl esters) and Nilsson et al, US 2008/0004344 (salts)) have been developed in an effort to overcome the deficiencies of current FAE (Fumaric acid ester) therapy. Controlled release pharmaceutical compositions comprising fumaric acid esters are disclosed by Nilsson and Muller, WO 2007/042034. Glycolamide ester prodrugs are described by Nielsen and Bundgaard, J Pharm Sci 1988, 77(4), 285-298.

Prodrugs of monomethyl fumarate and therapeutic uses thereof are disclosed in US Patent Publication US 2010/0048651 published Feb. 25, 2010.

Other prodrugs of monomethyl fumarate and therapeutic uses thereof are disclosed in US Patent Publication US 2013/0203753 published Aug. 8, 2013.

Joshi et al., US Patent Application Publication No. 2004/0054001, discloses using fumaric acid derivatives, such as monoalkyl and dialkyl fumarates, for treating cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma and Castleman tumors.

Kahrs US 2013/0172391 discloses the use of MMF and DMF for the treatment of diseases including, among others, chronic lymphocytic leukemia.

Wustrow et al. US 2014/0057918 discloses the use of MMF and prodrugs of MMF to treat a tumor.

NRF2 deficiency, demonstrated by NRF2 knockout in murine models, results in an earlier onset of cardiac dysfunction induced by pressure and volume overload (Li et al., Arterioscler Thromb Vasc Biol. 2009, 29(11), 1843-50). Certain NRF2 activators such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α estradiol have been used as therapeutic targets to reduce cardiac remodeling, but fatty acid fumarate derivatives have not been used yet to reduce cardiac remodeling (Zhou et al; J Appl Physiol. 2015, 119(8), 944-951).

Fumarates are cardioprotective in acute situations via activation of the NRF2 pathway in acute ischemia due to myocardial infarction (Ashrafian et. al; Cell Metab. 2012, 15(3), 361-71). However, Ashrafian et. al claims that fumarates are harmful in chronic situations, including heart failure.

Therefore, it is intended that the ability to provide the effects of fatty acids and fumarates in a synergistic way would provide benefits in treating heart failure diseases, including heart failure with preserved ejection fraction (HFPEF).

### SUMMARY

The present disclosure relates to methods and compositions useful in the treatment of heart failure diseases. The methods and compositions described herein comprise one or more FAFDs for the treatment of a heart failure disease.

In a first aspect, the heart failure disease is one of: heart failure with preserved ejection fraction (HFPEF); heart failure with ejection fraction ≥40%; diastolic heart failure; heart failure with elevated levels of TNF-α, IL-6, CRP, or TGF-β; hypertension with risk of developing HFPEF; atrial fibrillation with risk of developing HFPEF; diabetes with risk of developing HFPEF; COPD with risk of developing HFPEF; ischemic heart disease with risk of developing HFPEF; obesity with risk of developing HFPEF; chronic heart failure; compensated heart failure; and decompensated heart failure. In some embodiments, heart failure disease is heart failure with preserved ejection fraction.

FAFDs to be used in the treatment of HFPEF are molecular conjugates. A molecular conjugate comprises a fumarate and a fatty acid wherein the fatty acid is selected from the group consisting of omega-3 fatty acids, fatty acids that are metabolized in vivo to omega-3 fatty acids, and lipoic acid, and the conjugate is capable of hydrolysis to produce free fumarate and free fatty acid. In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid and lipoic acid. In other embodiments, the fatty acid is selected from eicosapentaenoic acid, docosahexaenoic acid and lipoic acid. In some embodiments, the hydrolysis is enzymatic.

The present disclosure provides methods of treating a heart failure disease, including HFPEF, by administering a FAFD of Formula (I) or (II), and pharmaceutical compositions containing a FAFD of Formula (I) or (II).

In another aspect, the methods disclosed herein use FAFDs of the Formula I and Formula II:

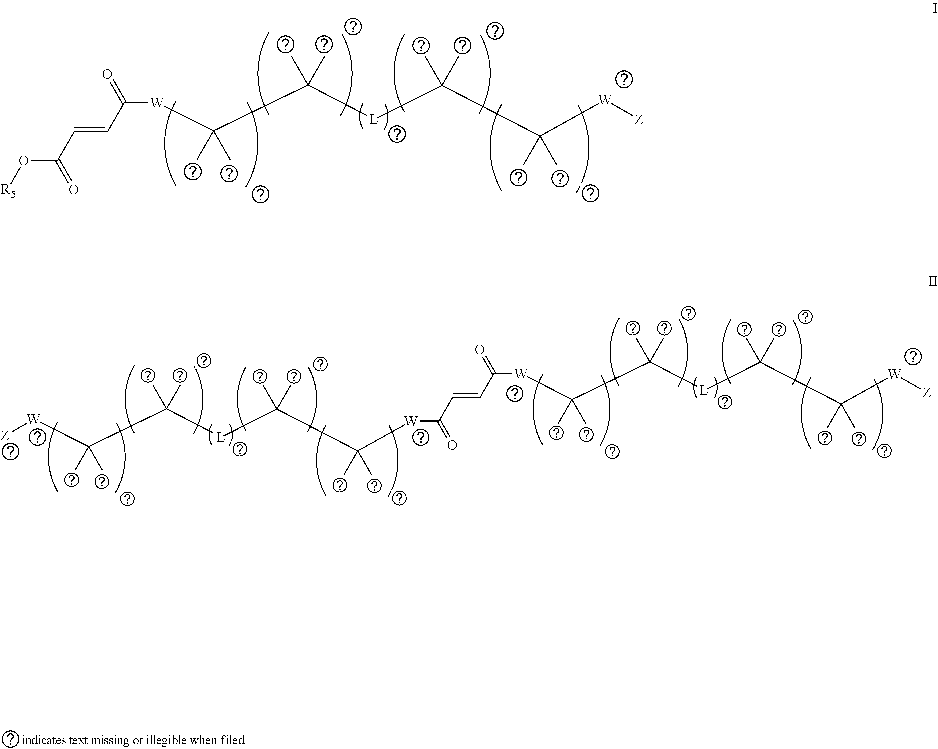

and pharmaceutically acceptable salts, hydrates, solvates, prodrugs, enantiomers, and stereoisomers thereof;

wherein

each W1, W2, W1′, and W2′ is independently null, O, S, NH, or NR, or W1 and W2, or W1′ and W2′ can be taken together to form an optionally substituted imidazolidine or piperazine group;

each a, b, c, d, a′, b′, c′, and d′ is independently —H, -D, —CH3, —OCH3, —OCH2CH3, —C(O)OR, —O—Z, or benzyl, or two of a, b, c, and d or any two of a′, b′, c′, and d′ can be taken together, along with the single carbon to which they are bound, to form a cycloalkyl or heterocycle;

each n, o, p, q, n′, o′, p′, and q′ is independently 0, 1, or 2;

each L and L′ is independently null, —O—, —C(O)—, —S—, —S(O)—, —S(O)2-, —S—S—, —(C1-C6 alkyl)-, —(C3-C6 cycloalkyl)-, a heterocycle, a heteroaryl,

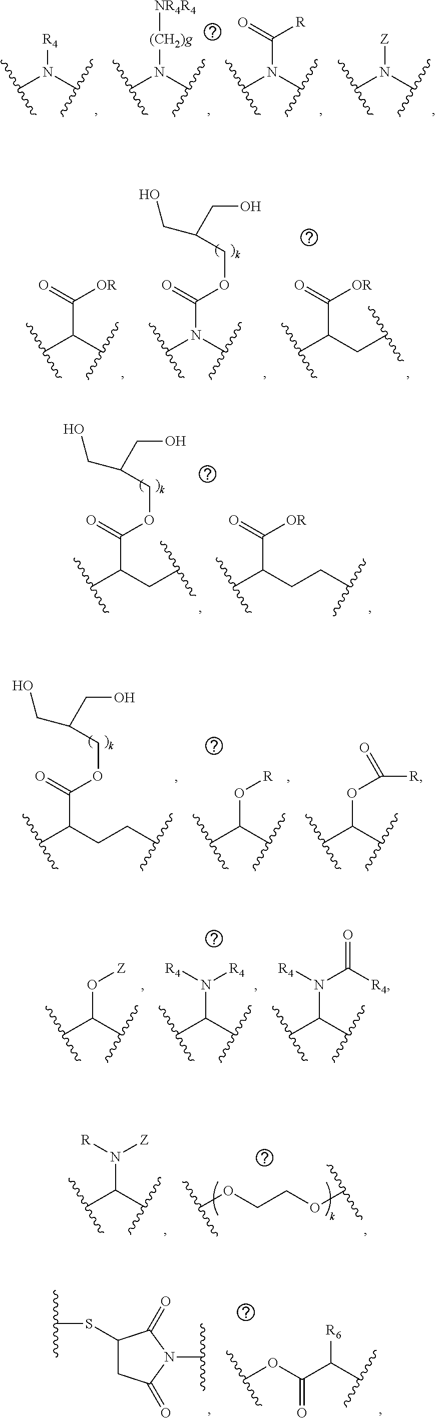

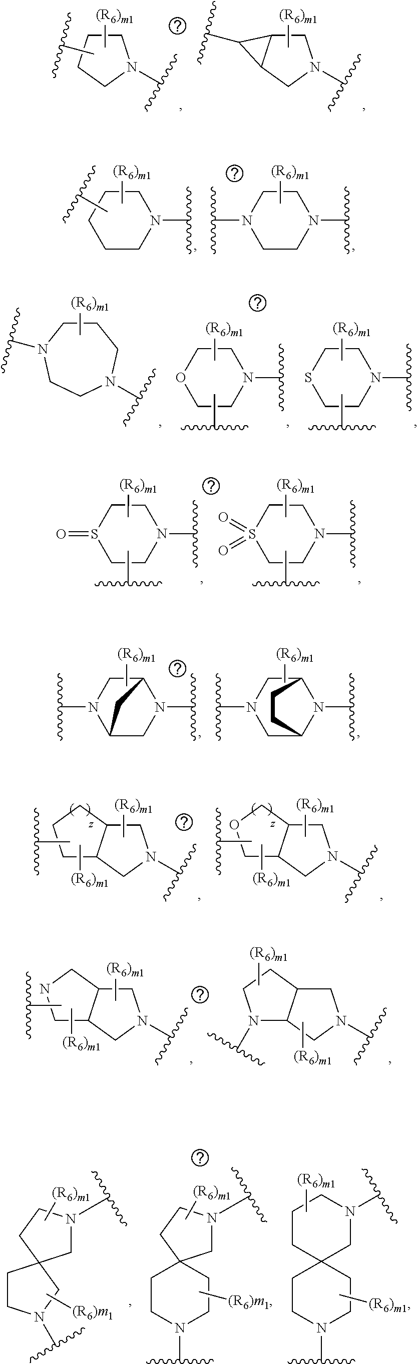

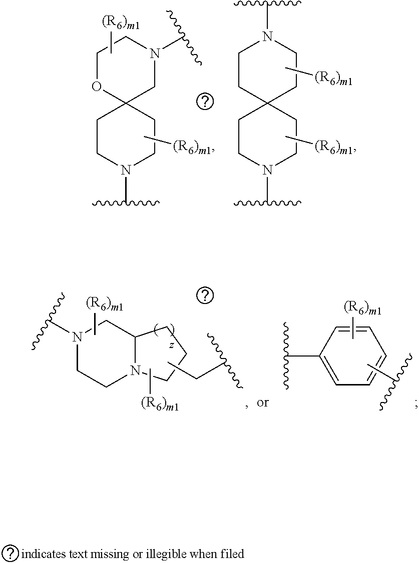

wherein the representation of L and L′ is not limited directionally left to right as is depicted, rather either the left side or the right side of L and L′ can be bound to the W1 or W1′ side of the compound of Formula I or Formula II, respectively;

each R6 is independently —H, -D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2, 3, or 4;

each h is independently 1, 2, 3, or 4;

each m and m′ is independently 0, 1, 2, or 3; if m or m′ is more than 1, then L or L′ can be the same or different;

each ml is independently 0, 1, 2, or 3;

k is 0, 1, 2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each Z and Z′ is independently H,

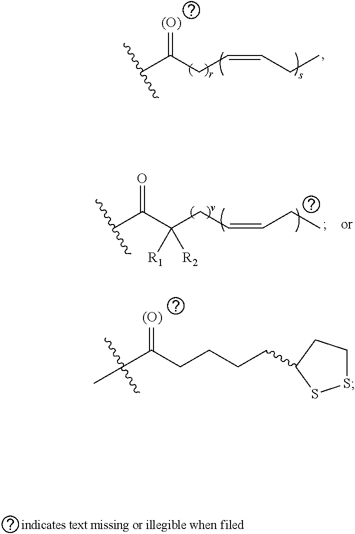

Provided that there is at least one of the following:

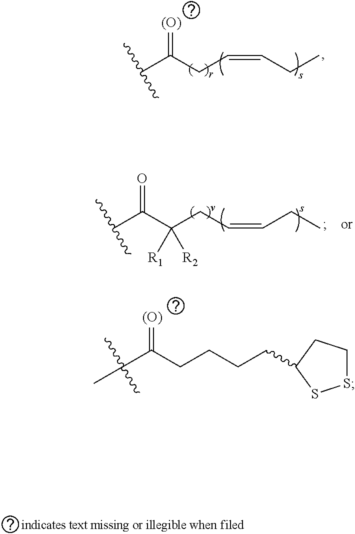

is in the compound;

each t is independently 0 or 1;

each r is independently 2, 3, or 7;

each s is independently 3, 5, or 6;

each v is independently 1, 2, or 6;

each R1 and R2 is independently —H, -D, —C1-C4 alkyl, -halogen, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H or straight or branched C1-C4 alkyl optionally substituted with OH or halogen;

provided that when each of m, n, o, p, and q, is 0, W1 and W2 is each null, and Z is

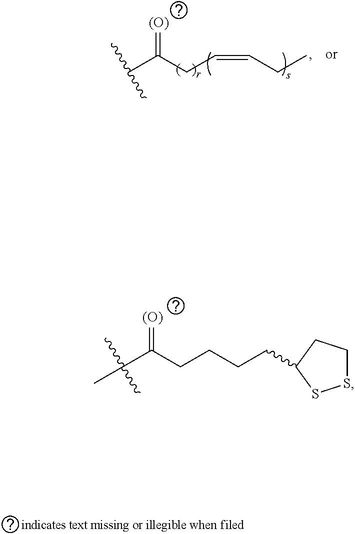

then t must be 0;

when each of m′, n′, o′, p′, and q′, is 0, W1′ and W2′ is each null, and Z′ is

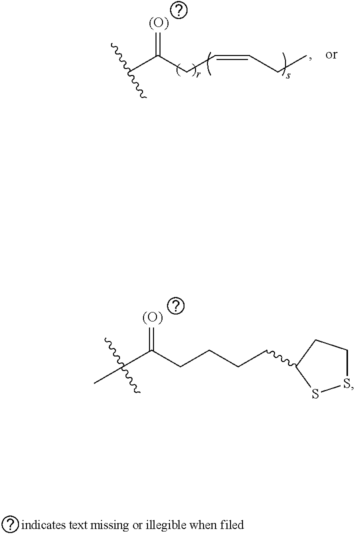

then t must be 0; and when each of m, n, o, p, and q is 0; and W1 and W2 is each null, or when each of m′, n′, o′, p′, and q′ is 0; W1′ and W2′ are each null; then Z or Z′ must not be

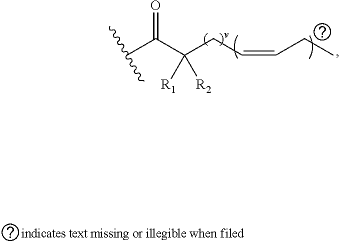

In another aspect, methods disclosed herein use FAFDs of Formula IA:

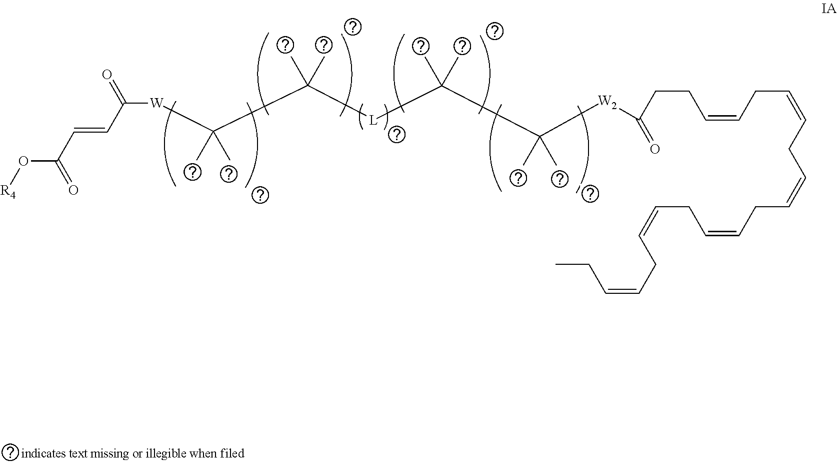

and pharmaceutically acceptable salts, hydrates, solvates, prodrugs, enantiomers, and stereoisomers thereof;

wherein each W1 and W2 is independently null, O, S, NH, or NR; or W1 and W2 can be taken together to form an optionally substituted imidazolidine or piperazine group;

each a, b, c, and d is independently —H, -D, —CH3, —OCH3, —OCH2CH3, —C(O)OR, or benzyl; or two of a, b, c, and d can be taken together, along with the single carbon to which they are bound, to form a cycloalkyl or heterocycle;

each n, o, p, and q is independently 0, 1, or 2;

each L is independently null, —O—, —C(O)—, —S—, —S(O)—, —S(O)2-, —S—S—, —(C1-C6 alkyl)-, —(C3-C6 cycloalkyl)-, a heterocycle, a heteroaryl,

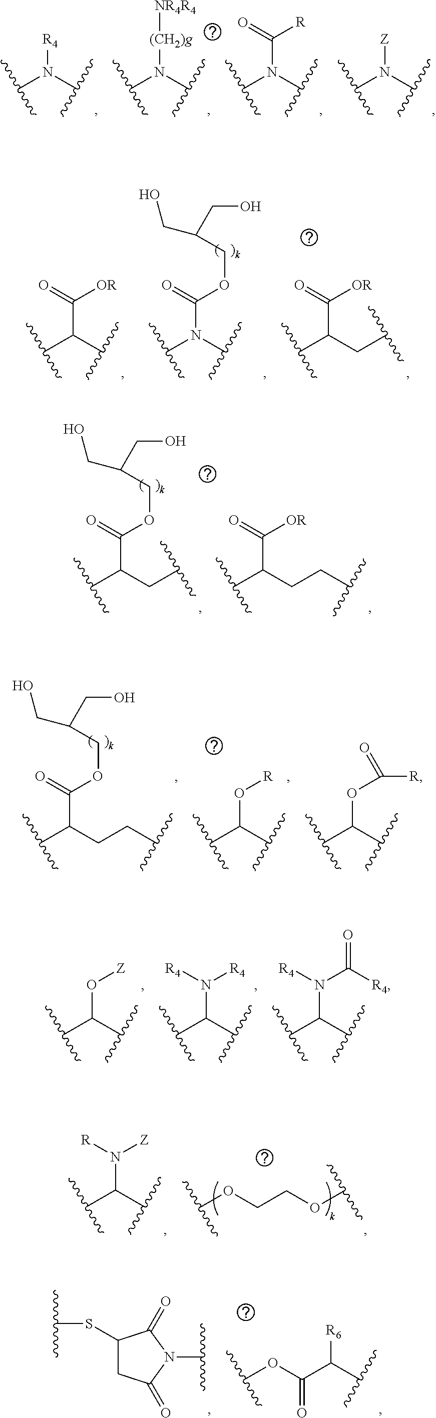

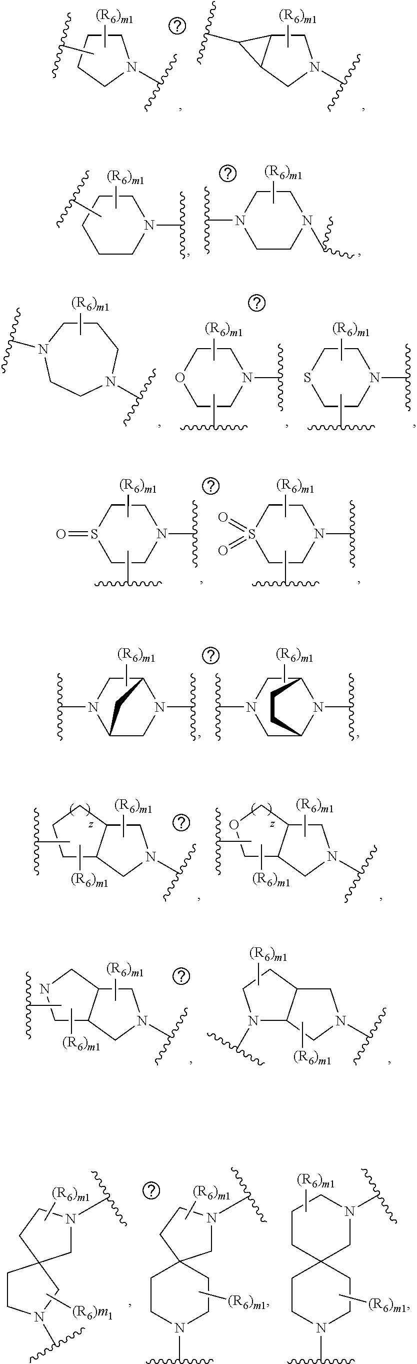

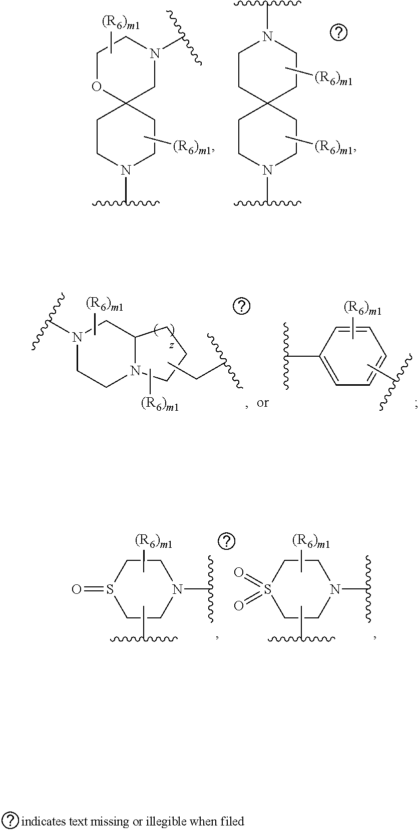

wherein the representation of L is not limited directionally left to right as is depicted, rather either the left side or the right side of L can be bound to the W1 side of the compound of Formula IA;

each R6 is independently —H, -D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2, 3, or 4;

each h is independently 1, 2, 3, or 4; each m is independently 0, 1, 2, or 3; if m is more than 1, then L can be the same or different;

each ml is independently 0, 1, 2, or 3;

k is 0, 1, 2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H, or straight or branched C1-C4 alkyl optionally substituted with OH, or halogen.

In another aspect, methods disclosed herein use FAFDs of Formula IB:

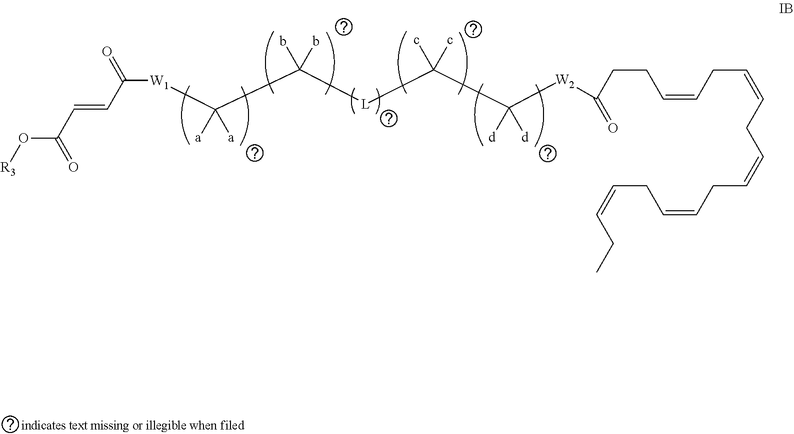

and pharmaceutically acceptable salts, hydrates, solvates, prodrugs, enantiomers, and stereoisomers thereof;

wherein

each W1 and W2 is independently null, O, S, NH, or NR; or W1 and W2 can be taken together can form an optionally substituted imidazolidine or piperazine group;

each a, b, c, and d is independently —H, -D, —CH3, —OCH3, —OCH2CH3, —C(O)OR, or benzyl; or two of a, b, c, and d can be taken together, along with the single carbon to which they are bound, to form a cycloalkyl or heterocycle;

each n, o, p, and q is independently 0, 1, or 2;

each L is independently null, —O—, —C(O)—, —S—, —S(O)—, —S(O)2-, —S—S—, —(C1-C6 alkyl)-, —(C3-C6 cycloalkyl)-, a heterocycle, a heteroaryl,

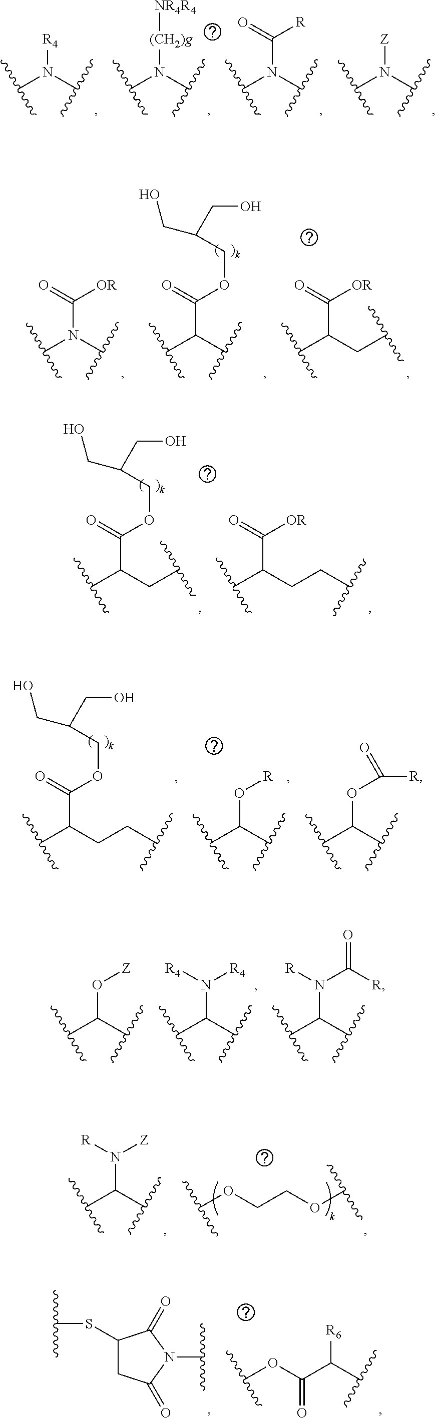

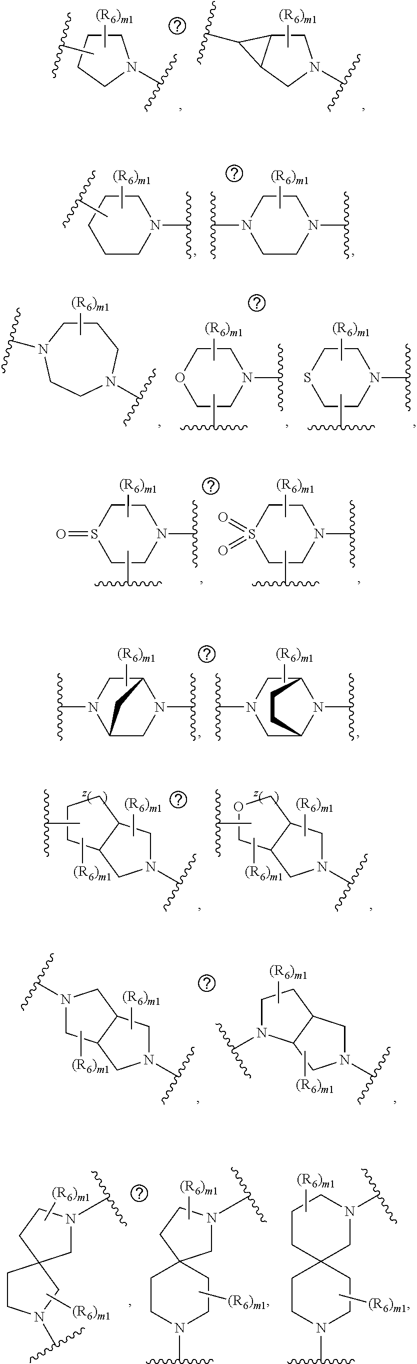

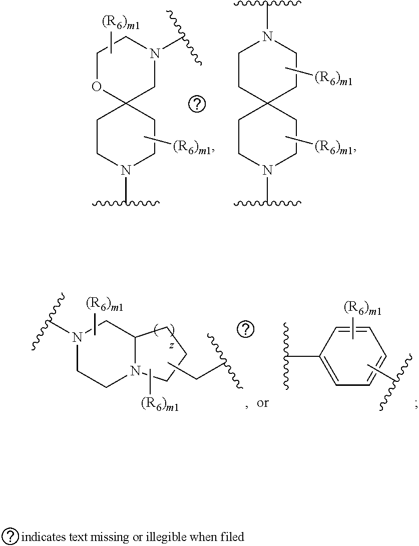

wherein the representation of L is not limited directionally left to right as is depicted, rather either the left side or the right side of L can be bound to the W1 side of the compound of Formula IB;

each R6 is independently —H, -D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2, 3, or 4;

each h is independently 1, 2, 3, or 4;

each m is independently 0, 1, 2, or 3; if m is more than 1, then L can be the same or different;

each ml is independently 0, 1, 2 or 3;

k is O, 1, 2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H, or straight or branched C1-C4 alkyl optionally substituted with OH, or halogen.

In another aspect, the methods disclosed herein use FAFDs of Formula IC:

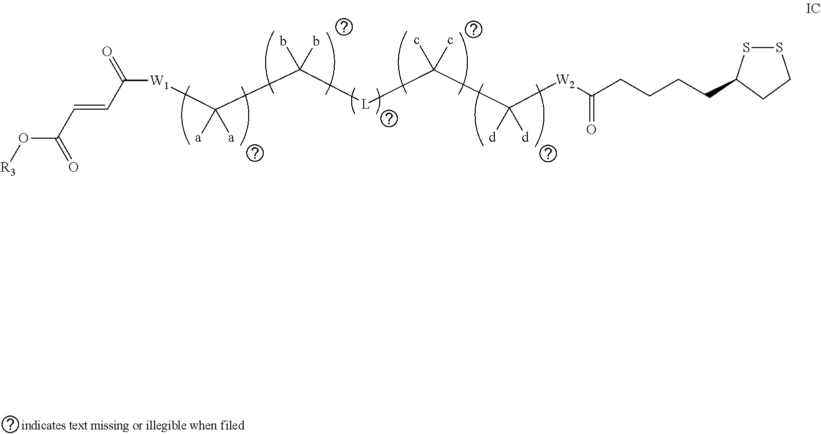

and pharmaceutically acceptable salts, hydrates, solvates, prodrugs, enantiomers, and stereoisomers thereof;

wherein

each W1 and W2 is independently null, O, S, NH, or NR, or W1 and W2 can be taken together can form an optionally substituted imidazolidine or piperazine group;

each a, b, c, and d is independently —H, -D, —CH3, —OCH3, —OCH2CH3, —C(O)OR, or benzyl; or two of a, b, c, and d can be taken together, along with the single carbon to which they are bound, to form a cycloalkyl or heterocycle;

each n, o, p, and q is independently 0, 1, or 2;

each L is independently null, —O—, —C(O)—, —S—, —S(O)—, —S(O)2-, —S—S—, —(C1-C6 alkyl)-, —(C3-C6 cycloalkyl)-, a heterocycle, a heteroaryl,

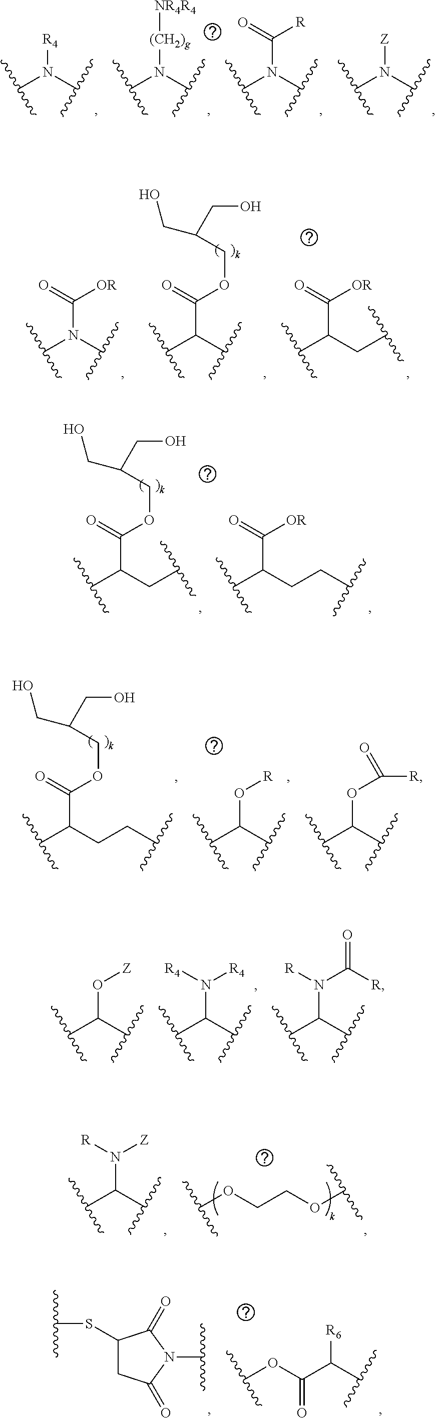

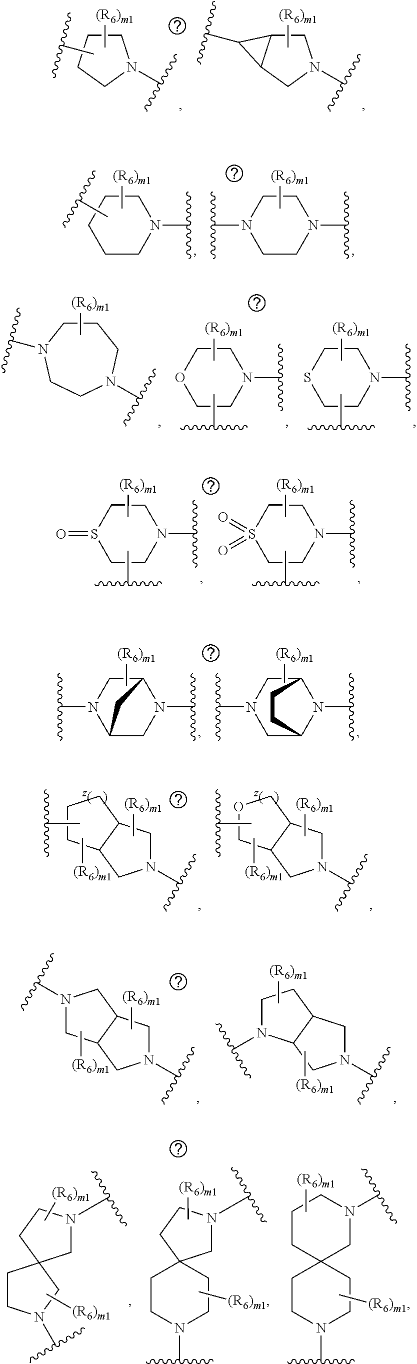

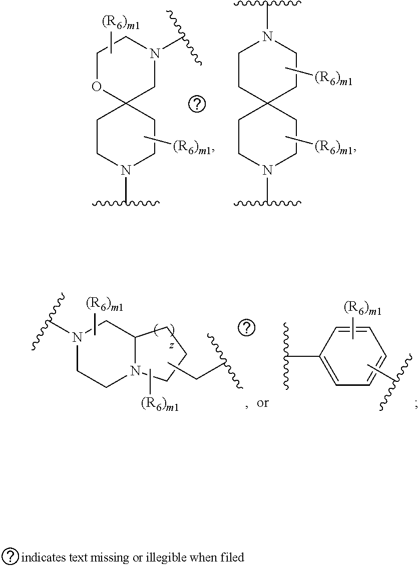

wherein the representation of L is not limited directionally left to right as is depicted, rather either the left side or the right side of L can be bound to the W1 side of the compound of Formula IC;

each R6 is independently —H, -D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2, 3, or 4;

each h is independently 1, 2, 3, or 4;

each m is independently 0, 1, 2, or 3; if m is more than 1, then L can be the same or different;

each ml is independently 0, 1, 2, or 3;

k is 0, 1, 2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H, or straight or branched C1-C4 alkyl optionally substituted with OH or halogen.

In compounds of Formula I, IA, IB, IC, and II, any one or more of H may be substituted with a deuterium. It is also understood that in compounds of Formula I, IA, IB, IC, and II, that a methyl substituent can be substituted with a C1-C6 alkyl.

In one embodiment of the present disclosure, methods disclosed herein use FAFD of formula (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof.

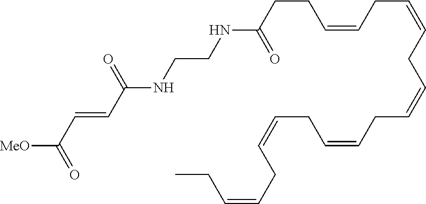 [SMILES: CC/C=C\C/C=C\C/C=C\C/C=C\C/C=C\C/C=C\CCC(=O)NCCNC(=O)/C=C/C(=O)OC]

The present disclosure also provides pharmaceutical formulations comprising at least one FAFD as described in U.S. Pat. No. 8,969,354, the disclosure of which is herein incorporated by reference in its entirety, and one or more pharmaceutically acceptable carriers for the treatment of heart failure disease. In some embodiments, the heart failure disease is heart failure with preserved ejection fraction (HFPEF).

In another embodiment, a pharmaceutical composition is administered to the subject, wherein said pharmaceutical composition comprises about 20 mg to about 5000 mg of (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof.

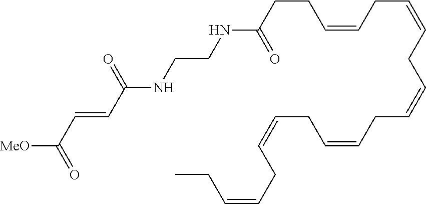 [SMILES: CC/C=C\C/C=C\C/C=C\C/C=C\C/C=C\C/C=C\CCC(=O)NCCNC(=O)/C=C/C(=O)OC]

In some embodiments, a pharmaceutical composition is administered to the subject, wherein said pharmaceutical composition comprises a therapeutically effective amount of (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof that is shown to provide MMF plasma exposure comparable to dimethyl fumarate (DMF) 120 mg to 720 mg per day.

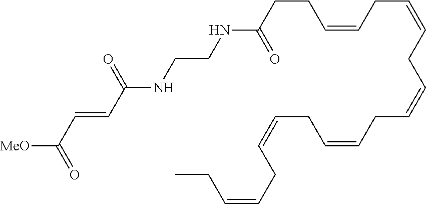 [SMILES: CC/C=C\C/C=C\C/C=C\C/C=C\C/C=C\C/C=C\CCC(=O)NCCNC(=O)/C=C/C(=O)OC]

In one embodiment, (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof is administered in combination with one or more second agents useful for treating heart failure. In various embodiment, the second agent is selected from the group consisting of: a diuretic, an ace-inhibitor, a beta-blocker, an angiotensin receptor blocker, isosorbide dinitrate, hydralazine, an angiotensin receptor-neprilysin inhibitor, an aldosterone antagonist, a PDE5 inhibitor, a statin, a neprilysin inhibitor, an aldosterone inhibitor, and an antitumor necrosis factor-alpha therapy. In one embodiment, the second agent is a statin.

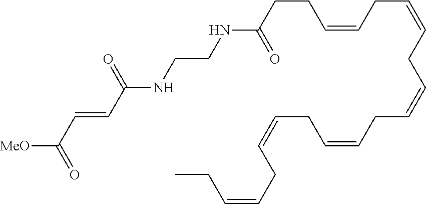 [SMILES: CC/C=C\C/C=C\C/C=C\C/C=C\C/C=C\C/C=C\CCC(=O)NCCNC(=O)/C=C/C(=O)OC]

Another aspect of the present disclosure provides for a pharmaceutical composition comprising (a) (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt and (b) a statin and one or more pharmaceutically acceptable excipients.

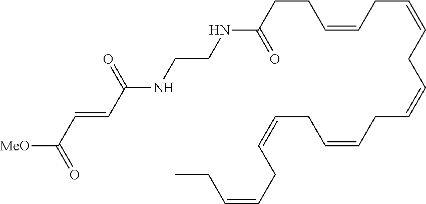 [SMILES: CC/C=C\C/C=C\C/C=C\C/C=C\C/C=C\C/C=C\CCC(=O)NCCNC(=O)/C=C/C(=O)OC]

In some embodiments, the pharmaceutical composition comprises (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof at a dose range of about 20 mg to about 5000 mg of the FAFD per day. Compositions for in vivo or in vitro use can contain about 20, 50, 75, 100, 150, 250, 500, 750, 1000, 1250, 2500, 3500, or 5000 mg of the FAFD. In one embodiment, the composition is in the form of a tablet that can be scored. Effective plasma levels of the FAFD can range from about 0.002 mg to about 100 mg per kg of body weight per day, for example as described in U.S. patent application Ser. No. 15/075,829, the disclosure of which is incorporated by reference in its entirety.

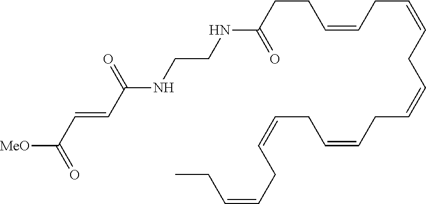 [SMILES: CC/C=C\C/C=C\C/C=C\C/C=C\C/C=C\C/C=C\CCC(=O)NCCNC(=O)/C=C/C(=O)OC]

Another aspect of the disclosure provides a method of treating a heart failure disease in a subject in need thereof, the method comprising: administering to the subject a therapeutically effective amount of (a) (E)-methyl 4-(2-(4Z,7Z,10Z,13Z,16Z,19Z)-docosa-4,7,10,13,16,19-hexaenamidoethylamino)-4-oxobut-2-enoate (I-1) and either separately or together with (b) a statin.

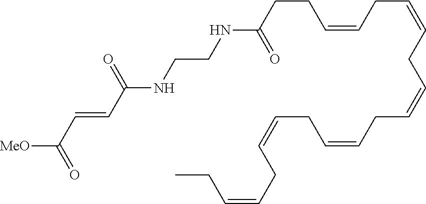 [SMILES: CC/C=C\C/C=C\C/C=C\C/C=C\C/C=C\C/C=C\CCC(=O)NCCNC(=O)/C=C/C(=O)OC]

Another aspect of the present disclosure is directed to methods useful in the treatment of heart failure diseases including, heart failure with preserved ejection fraction (HFPEF), the method comprising administering to the subject a therapeutically effective amount of a compound that upregulates the Nrf2 pathway, inhibits NF-κB, or increases adiponectin. Compositions described herein comprise one or more dialkyl fumarates that are configured for the treatment of a heart failure disease including, heart failure with preserved ejection fraction. In one embodiment, dialkyl fumarate is dimethyl fumarate. In some embodiments, the disclosed pharmaceutical preparations are in the form of micro-tablets or micro-pellets containing dialkyl fumarates.

Additionally, methods disclosed herein provide for administering pharmaceutical preparations containing dialkyl fumarates in combination with one or more second agents that do not upregulate the Nrf2 pathway, inhibit NF-κB, or increase adiponectin for use in the treatment of a heart failure disease. In some embodiments, the second agent may include one or more of: a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy and combination thereof. In one embodiment, the second agent is a statin.

The present disclosure provides for the treatment of a heart failure disease by the administration of one or more dialkyl fumarates of the formula:

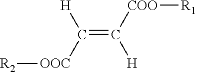

wherein R1 and R2, which may be the same or different, independently represent a linear, branched, or cyclic, saturated or unsaturated C1-20 alkyl radical which may be optionally substituted with halogen (Cl, F, I, Br), hydroxy, alkoxy, nitro, or cyano.

The C1-20 alkyl radicals, preferably C1-8 alkyl radicals, most preferably C1-5 alkyl radicals are, for example, methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxyethyl, 2 or 3-hydroxy propyl, 2-methoxy ethyl, methoxy methyl or 2- or 3-methoxy propyl. In one embodiment, at least one of the radicals or R2 is C1-5 alkyl, especially methyl or ethyl. In one embodiment, R1 and R2 are the same or different C1-5 alkyl radicals such as methyl, ethyl, n-propyl, or t-butyl. In one embodiment, R1 and R2 are identical and are methyl or ethyl. In one embodiment, one or more dialkyl fumarates are dimethyl fumarate, methylethyl fumarate, and diethyl fumarate.

The present disclosure provides method for chronic treatment of heart failure with preserved ejection fraction (HFPEF) by administering to the subject, over the long term or over a prolonged period of time, a daily dose of dimethyl fumarate.

In one embodiment, dimethyl fumarate is administered to the subject in a therapeutically effective amount of about 120 to 720 mg per day and in separate or the same administrations over the long term or over a prolonged period of time.

The present disclosure provides a method for treating heart failure with preserved ejection fraction by a combination therapy, the method comprising: (a) providing simultaneously, separately, or sequentially to a subject a therapeutically effective amount of a first compound that upregulates the Nrf2 pathway, inhibits NF-κB, or increases adiponectin and (b) administering a second agent that does not upregulate the Nrf2 pathway, inhibit NF-κB, or increase adiponectin. In some embodiments, the first compound is dimethyl fumarate and the second agent selected from the group consisting of: a diuretic, an ace-inhibitor, a beta-blocker, an angiotensin receptor blocker, isosorbide dinitrate, hydralazine, an aldosterone inhibitor, an angiotensin receptor neprilysin inhibitor, a PDE5 inhibitor, a statin, a neprilysin inhibitor, an antitumor necrosis factor-alpha therapy as active ingredients, and a combination thereof. In one embodiment, dimethyl fumarate is administered at a dose of 120 mg to 720 mg and a statin is given at a dose of 10 mg to 80 mg.

The present disclosure provides for pharmaceutical preparations used for treatment of heart failure with preserved ejection fraction. The pharmaceutical preparation may include an active ingredient and optionally carriers and excipients. In one embodiment, the active ingredient includes dimethyl fumarate and a second agent selected from group consisting of a beta-blocker, an ace-inhibitor, an angiotensin receptor blocker, isosorbide dinitrate, hydralazine, an aldosterone inhibitor, an angiotensin receptor neprilysin inhibitor, a PDE5 inhibitor, a statin, a neprilysin inhibitor, an antitumor necrosis factor-alpha therapy and combinations thereof; and pharmaceutical preparation is in the form of microtablets and the mean diameter of the microtablets is about 2,000 μm, excluding any coating on the microtablets. In another embodiment, the pharmaceutical preparation includes dimethyl fumarate at a dose of 10 mg to 300 mg and statin at a dose of 10 mg to 80 mg.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION

### Section I: Dialkyl Fumarates

The present disclosure relates to the fields of cardiology and pharmaceutical sciences. In particular, the present disclosure relates to certain pharmaceutical preparations comprising dialkyl fumarates and administering said pharmaceutical preparations alone or in combination with one or more second agents for the treatment of a heart failure disease including, heart failure with preserved ejection fraction.

Compositions described herein comprise one or more dialkyl fumarates configured for the treatment of a heart failure disease. In some embodiments the compositions are used for the prevention or treatment of heart failure with preserved ejection fraction (HFPEF). In some embodiments, the disclosed pharmaceutical preparations are in the form of micro-tablets or micro-pellets containing dialkyl fumarates.

The present disclosure provides, in part, methods for the prevention or treatment of a heart failure disease by administering to a subject in need thereof, a therapeutically effective amount of one or more pharmaceutical preparations in the form of micro-tablets or micro-pellets containing dialkyl fumarates.

The heart failure disease may be heart failure with preserved ejection fraction (HFPEF); heart failure with ejection fraction ≥40%; diastolic heart failure; heart failure with elevated levels of TNF-α, IL-6, CRP, or TGF-β; hypertension with a risk of developing HFPEF; atrial fibrillation with a risk of developing HFPEF; diabetes with a risk of developing HFPEF; COPD with a risk of developing HFPEF; ischemic heart disease with a risk of developing HFPEF; obesity with a risk of developing HFPEF; chronic heart failure; compensated heart failure; decompensated heart failure; or other conditions known to have a high risk of developing HFPEF

Patients with heart failure can be divided into those with (1) heart failure with a reduced ejection fraction (HFREF) and (2) heart failure with a preserved ejection fraction (HFPEF). All of these patients, regardless of ejection fraction status (EF value), have the clinical syndrome of heart failure. In addition, many features are similar across the EF spectrum, including frequent hospitalization and reduced survival. Patients with HFPEF have a devastating 5-year mortality rate (approaching 60%), costly morbidity (6-month hospitalization rate of 50%), and debilitating symptoms (maximum myocardial oxygen consumption [MVo₂] averaging 14 mL/g/min). Clear differences are also recognized between HFPEF and HFREF. Compared with those with HFREF, patients with HFPEF are typically older and more likely to be female; however, HFPEF does occur in both men and women throughout the fifth to the ninth decades of life. The most common disease leading to HFPEF is systolic hypertension, which is present in more than 85% of patients. Differences in cardiovascular structure and function between HFPEF and HFREF also are well recognized. Patients with HFPEF have normal LV end-diastolic volume and normal (or near-normal) EF and stroke volume and commonly exhibit concentric remodeling of either the left ventricular (LV) chamber and/or cardiomyocytes. Finally, differences are also evident in the effects of pharmacologic treatment in patients with HFREF versus HFPEF. Standard heart failure therapy shown to be effective in HFREF has not been found to reduce morbidity or mortality associated with HFPEF, leaving a substantial area of unmet need, which the present disclosure addresses with the use of dialkyl fumarates, such as dimethyl fumarate (DMF), to treat or prevent heart failure diseases.

Drugs that have previously shown mortality benefit for HFREF but not for HFPEF include beta-blockers, ACE-inhibitors, angiotensin receptor blockers, isosorbide dinitrate, hydralazine, angiotensin receptor-neprilysin inhibitors, and aldosterone antagonists. While there are many drugs available to treat patients with HFREF, the only strategy for HFPEF remains managing volume status with diuretics and managing co-morbidity conditions. Due to lack of therapeutic options, HFPEF remains deadly.

The pathophysiology of HFPEF is still not fully understood. A recent hypothesis of HFPEF, as described by Zuo et al, traces its roots to a proinflammatory state initiated in part by the existence of comorbidities (e.g., hypertension, diabetes mellitus, vasculopathy, renal disease, metabolic syndrome, and atrial fibrillation) that create a favorable environment for the production of reactive oxygen species (ROS). Triggering a cascade that involves reduced nitric oxide (NO) availability and elevated ROS levels in the coronary endothelium that eventually contribute to hypertrophy and increased resting tension in cardiomyocytes (J Appl Physiol. 2015, 119(8), 944-951). In HFPEF patients, ROS produced during comorbidity-induced endothelial inflammation may trigger a signaling cascade involving NO that ultimately increases interstitial fibrosis and activates cardiac remodeling. These actions contribute to the hallmarks of HFPEF: ventricular stiffness, impaired relaxation and cardiac dysfunction.

Based on Zuo et al hypothesis of inflammation as a culprit in HFPEF, a number of treatments to reduce inflammation have been explored. Anti-inflammatory treatments including antitumor necrosis factor-alpha therapies and 3-hydroxy-3-methylglutaryl coenzyme A reductase inhibitors (statins) have in general failed to improve clinical outcomes. Although a small retrospective, nonrandomized study suggested potential benefits to statin treatment in HFPEF, no benefit was seen in the subgroup of patients with preserved EF enrolled in the large, prospective GISSI-HF (i.e., Gruppo Italiano per lo Studio della Sopravvivenza nell'Infarto Miocardico—Heart Failure) trial, which randomized patients to rosuvastatin or placebo. Angiotensin receptor blockers (ARB) also reduce markers of inflammation and improve endothelial function, but they have been ineffective in improving outcomes in HFPEF patients in randomized trials.

Paulus and Tschope (J Am Coll Cardiol, 2013, 62, 263-271) postulate that comorbidities in HFPEF (e.g., obesity, hypertension, diabetes, chronic obstructive pulmonary disease, anemia, and chronic kidney disease) conspire to create a systemic inflammatory state that promotes coronary microvascular endothelial dysfunction. Endothelial inflammation results in generation of reactive oxygen species and reduces NO bioavailability, which in turn results in lower levels of cyclic guanosine monophosphate (cGMP) and lower activity of protein kinase G (PKG). Declining PKG activity accelerates pro-hypertrophic signaling and increases myocyte stiffness by promoting hypophosphorylation of titin, enhancing diastolic dysfunction and ventricular stiffening characteristics of HFPEF.

Based on this hypothesis, it was hypothesized that sildenafil, which raises myocardial PKG activity by inhibiting breakdown of cGMP by phosphodiesterase 5, may improve pulmonary artery pressures, right ventricular function, and LV relaxation and distensibility in HFPEF. However, sildenafil did not impact the primary outcome of functional capacity in HFPEF patients in the RELAX trial (i.e., Phosphodiesterase-5 Inhibition to Improve Clinical Status and Exercise Capacity in Diastolic Heart Failure). Thus, there still remains an urgent need for new therapies to treat HFPEF.

Another possible candidate for treating HFPEF is LCZ696, a combined angiotensin receptor neprilysin inhibitor (ARNI) that has been recently shown to reduce mortality in HFREF but not yet in HFPEF patients. LCZ696 inhibits natriuretic peptide breakdown and enhances cGMP activation, and in HFPEF was associated with incremental reductions in circulating N-terminal pro-B-type natriuretic peptide (NT-proBNP) levels when compared to treatment with the ARB valsartan alone. However, these reductions were incremental, and it is yet to be seen whether LCZ696 or other angiotensin receptor-neprilysin inhibitors will lead to any significant mortality or clinical benefit in HFPEF patients.

Another HFREF drug spironolactone, an aldosterone antagonist, was shown to be ineffective for HFPEF in the TOPCAT trial (i.e., Treatment of Preserved Cardiac Function Heart Failure with an Aldosterone Antagonist). In 3,445 patients with symptomatic heart failure and a preserved ejection fraction, treatment with spironolactone did not significantly reduce the incidence of the primary composite outcome of death from cardiovascular causes, cardiac arrest, or hospitalization for the management of heart failure.

Thus, despite many attempts to treat the deadly HFPEF condition, a beneficial treatment to improve HFPEF remains elusive. The present disclosure fills this unmet need by providing a novel treatment for patients with a heart failure disease, including HFPEF.

The present disclosure is directed to the surprising and unexpected discovery that using dialkyl fumarates, alone or in combination with one or more second agents, is beneficial for the treatment of a heart failure disease. The object of the present disclosure is achieved by the use of certain pharmaceutical preparations comprising dialkyl fumarates and administering said pharmaceutical preparations alone or in combination with one or more second agents for the treatment of a heart failure disease. In some embodiments, the compositions are used for the prevention or treatment of heart failure with preserved ejection fraction (HFPEF). In some embodiments, the disclosed pharmaceutical preparations are in the form of micro-tablets or micro-pellets containing dialkyl fumarates.

In some embodiments, at least one dialkyl fumarate is combined with one or more compounds that reduce ROS, increase the availability of NO, increase cGMP, or increase PGK.

In some embodiments, at least one dialkyl fumarate is combined with one or more second agents selected from group consisting of a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy, and combination thereof.

In some embodiments, pharmaceutical preparations in the form of micro-tablets and micro-pellets comprising dialkyl fumarates are administered in combination with one or more compounds that reduce ROS, increase the availability of NO, increase cGMP, or increase PGK.

In some embodiments, pharmaceutical preparations in the form of micro-tablets and micro-pellets comprising dialkyl fumarates administered in combination with a diuretic, an ace-inhibitor, a beta-blocker, an angiotensin receptor blocker (ARB), isosorbide dinitrate, hydralazine, an angiotensin receptor-neprilysin inhibitor (ARNI), an aldosterone antagonist, a PDE5 inhibitor, a statin, a neprilysin inhibitor, an aldosterone inhibitor, an antitumor necrosis factor-alpha therapy, or a combination thereof, in order to treat or prevent a heart failure disease, including HFPEF.

Fumaric acid esters (FAEs), such as dialkyl fumarates like DMF, are approved for the treatment of psoriasis and multiple sclerosis, and have been proposed for use in treating a number of immunological, autoimmune, and inflammatory diseases. However, until the present disclosure, fumaric acid esters (FAEs), such as dialkyl fumurates like DMF, have not been explored as a therapy for HFPEF and other heart failure diseases.

FUMADERM®, an enteric coated tablet containing a salt mixture of monoethyl fumarate and dimethyl fumarate (DMF) which is rapidly hydrolyzed to monomethyl fumarate, regarded as the main bioactive metabolite, was approved in Germany in 1994 for the treatment of psoriasis. FUMADERM® is dosed three times a day (TID) with 1-2 grams/day administered for the treatment of psoriasis. FUMADERM® exhibits a high degree of interpatient variability with respect to drug absorption, and food strongly reduces bioavailability. Absorption is thought to occur in the small intestine with peak levels achieved 5-6 hours after oral administration. Significant side effects occur in 70-90% of patients (Brewer and Rogers, Clin Expt'l Dermatology 2007, 32, 246-49; and Hoefnagel et al., Br J Dermatology 2003, 149, 363-369). Side effects of current FAE therapy include gastrointestinal upset including nausea, vomiting, diarrhea, and/or transient flushing of the skin.

Dimethyl fumarate (DMF) is the active component of BG-12, also known as Tecfidera®, studied for the treatment of relapsing-remitting MS (RRMS). In a Phase III RRMS study, BG-12 significantly reduced the proportion of patients who had a relapse, the annualized relapse rate, the rate of disability progression, and the number of lesions on MRI.

The exact mechanisms by which DMF exerts its clinical efficacy in multiple sclerosis is unknown, but some of these effects are believed to be mediated through activation of the nuclear factor (erythroid-derived 2)-like 2 (NRF2) pathway, an endogenous defense mechanism against toxic cell stress. Under basal conditions, NRF2 is sequestered in the cytoplasm by the actin-associated protein kelch-like ECH-associated protein 1 (KEAP1), which targets NRF2 for ubiquitination and subsequent proteasomal degradation. However, in the presence of electrophiles or oxidative stress, these molecules can bind KEAP1 cysteine residues resulting in an allosteric conformational change that diminishes KEAP1-dependent degradation of NRF2. This allows NRF2 to accumulate and translocate to the nucleus, where NRF2 binds to the antioxidant responsive element (ARE), a cis-acting regulatory element that increases expression of detoxifying enzymes and antioxidant proteins. Various synthetic and naturally occurring compounds possessing electrophilic properties, including DMF, have been shown to modify specific cysteine residues on KEAP1 and subsequently activate NRF2.

One explanation for the beneficial effects of BG-12 in multiple sclerosis is that one pathway of FAEs, such as dialkyl fumarates like DMF, is the upregulation of NRF2, which increases expression of ARE, which increases expression of detoxifying enzymes and antioxidant proteins.

Along with research supporting NRF2 activation as a cytoprotective mechanism of DMF, other studies have hypothesized indirect regulation of NRF2 or NRF2-independent mechanisms of action for DMF, including activation of the hydroxycarboxylic acid receptor 2 (HCA2), inhibition of nuclear factor kappa-light-chain-enhancer of activated B cells (NF-κB), activation of hypoxia-inducible factor 1-alpha (HIF1A) and modulation of cellular glutathione (GSH). As α, β carboxylic acid unsaturated esters, fumarates are capable of interacting with various free cysteine residues by Michael addition including those present on the antioxidant thiol, GSH. Schmidt and colleagues have shown that DMF can stably bind GSH and rapidly deplete circulating levels. Other groups have suggested that this intracellular depletion contributes to the anti-inflammatory, immunosuppressive, and cytoprotective properties of DMF. It is therefore likely that multiple mechanisms underlie the immunomodulatory and neuroprotective effects of DMF, including but not limited to NRF2/ARE activation.

As described by Mudd and Kass, there is a preference for oxidation of fatty acids over glucose in a healthy heart (Nature, 2008; 451, 919-928). When the heart is pressure or volume overloaded, there may be a shift towards increased glucose oxidation, which is coupled to a decline in production of the transcriptional coactivator PGC1α. PGC1α modulates the expression of the transcription factors PPAR-α, ERR-α, NRF1, and NRF2, which affect mitochondrial biogenesis and fatty-acid oxidation.

As described by Li et al, NRF2 deficiency, demonstrated by NRF2 knockout in murine models, results in an earlier onset of cardiac dysfunction induced by pressure and volume overload (Arterioscler Thromb Vasc Biol. 2009, 29(11), 1843-50).

Zhou et al describes the role of the NRF2-mediated pathway in cardiac remodeling and heart failure (HF). HF and many of the conditions that predispose one to HF are associated with oxidative stress. Increased generation of reactive oxygen species (ROS) in the heart can directly lead to increased necrosis and apoptosis of cardiomyocytes, which subsequently induce cardiac remodeling and dysfunction. NRF2 is a transcription factor that controls the basal and inducible expression of a battery of antioxidant genes and other cytoprotective phase II detoxifying enzymes that are ubiquitously expressed in the cardiovascular system. NRF2 and its target genes may be critical regulators of cardiovascular homeostasis via the suppression of oxidative stress, which is the key player in the development and progression of HF. Zhou et al proposes certain NRF2 activators as therapeutic targets to reduce cardiac remodeling such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α-estradiol, but does not include FAEs, dialkyl fumurates, or DMF as revealed by the present disclosure.

The present disclosure discloses the use of fumaric acid esterases (FAEs), such as dialkyl fumarates like DMF, for the treatment of a heart failure disease, including HFPEF.

In acute ischemia due to myocardial infarction, Ashrafian et. al proposes that fumarates are cardioprotective in acute situations via activation of the NRF2 pathway (Cell Metab. 2012, 15(3), 361-71). However, Ashrafian et al claim that fumarates are harmful in chronic situations, including heart failure. The present disclosure counters Ashrafian et al. The present disclosure has determined that FAEs, such as dialkyl fumarates like DMF, may be surprisingly beneficial in the treatment of heart failure diseases, including HFPEF over the long term or during a prolonged period of time with a daily dose.

In some embodiments, long term administration means delivery of the drug in a regular interval during a pre-determined length or for as long as the subject's condition requires the drug. In some embodiments, the long term administration means administration with a drug holiday, for example, a daily dose given for one month, then two weeks off, and then a daily dose give for one month, etc.

Bardoxolone Methyl, also known as RTA 402, CDDO-methyl ester, and CDDO-Me, also is an activator of the NRF2 pathway and inhibits NF-κB. Bardoxolone Methyl was tested in humans as a treatment for chronic kidney disease (CKD) but was found to increase rates of heart failure, and the trial was stopped. Thus, it is surprising that DMF, a NRF2 activator, may be an effective therapy for treatment of HFPEF.

Further, Fumaderm was tested in a short term study (16 weeks) in psoriasis patients and was surprisingly found to increase adiponectin, a protein involved in regulating glucose levels as well as fatty acid breakdown (Schmieder et al., “Impact of fumaric acid esters on cardiovascular risk factors and depression in psoriasis: a prospective pilot study. Arch Dermatol Res, 2015, 307: (5), 413-424). Adiponectin may have cardioprotective properties, but such benefits are still hypothetical. In animal models, adiponectin deficiency has shown to worsen HFPEF, but it is still unknown whether increasing adiponectin will improve outcomes in HFPEF in humans (Tanaka et al., “Effects of Adiponectin on Calcium-Handling Proteins in Heart Failure With Preserved Ejection Fraction.” 2014 Circ Heart Fail, 7, 976-985.). Thus DMF, which increases adiponectin, may provide a therapeutic benefit in HFPEF.

Furthermore, pro-inflammatory cytokines IL-6 and TNF-α are raised in HFPEF, which may lead to increased activity of VCAM, E-Selection, and NADPH oxidase, which increase ROS in coronary microvasculature endothelial cells, leading to the hallmarks of HFPEF: ventricular stiffness, impaired relaxation, and cardiac dysfunction. In some embodiments, treating heart failure as described herein includes treating heart failure by reducing ventricular stiffness, increasing ventricular relaxation, and/or reducing cardiac dysfunction. The present disclosure includes that DMF may reduce damage of ROS in heart failure by multiple pathways including increasing the NRF2/ARE pathway, and possibly by reducing NFkB, which reduces IL-6 and TNF-α.

The present disclosure includes the use of FAEs, such as dialkyl fumarates like DMF, for the treatment of a heart failure disease. The full mechanism of fumaric acids is not completely understood, however, one pathway includes increased activity of the NRF2/ARE pathway. The present disclosure is directed to the surprising and unexpected discovery that using dialkyl fumarates (e.g., those that upregulate the Nrf2 pathway, inhibit NFκB, or increase adiponectin), alone or in combination with other compounds (e.g., those that do not upregulate the Nrf2 pathway, inhibit NFκB, or increase adiponectin), is beneficial for the therapy of a heart failure disease.

The object of the present disclosure is achieved by the use of certain pharmaceutical preparations comprising dialkyl fumarates and administering said pharmaceutical preparations for the treatment of a heart failure disease. In some embodiments the compositions are used for the prevention or treatment of heart failure with preserved ejection fraction (HFPEF). In some embodiments, the disclosed pharmaceutical preparations are in the form of micro-tablets or micro-pellets containing dialkyl fumarates.

The preparations according to the present disclosure do not contain any free fumaric acids per se.

Fumaric acid, for example, inhibits the growth of the Ehrlich ascites tumor in mice, reduces the toxic effects of mitomycin C and aflatoxin, and displays anti-psoriatic and anti-microbial activity. When administered parenterally, transdermally, and especially perorally, high dosages of fumaric acid or its derivatives known so far such as dihydroxyl fumaric acid, fumaramide, and fumaronitrile have such unacceptably severe side effects and high toxicity that, in most cases, such a therapy had to be abandoned in the past.

European Patent Application 0188 749, the disclosure of which is incorporated by reference in its entirety, already describes fumaric acid derivatives and pharmaceutical compositions containing the same for the treatment of psoriasis. Pharmaceutical compositions for the treatment of psoriasis containing a mixture of fumaric acid and other fumaric acid derivatives are known from DE-A-25 30 372, the disclosure of which is incorporated by reference in its entirety.

DE-A-26 21 214, the disclosure of which is incorporated by reference in its entirety, describes medicaments containing the fumaric acid monoethyl ester and its mineral salts as active ingredients for the treatment of psoriasis. The publication “Hautarzt (Dermatologist) (1987) 279-285” discusses the use of fumaric acid monoethyl ester salts. Pharmaceutical preparations containing a mixture of fumaric acid monoalkyl ester salts and a fumaric acid diester for the treatment of psoriasis, psoriatic arthritis, neurodermatitis, and enteritis regionalis Crohn are known from EP 0 312 697 B 1 the disclosure of which is incorporated by reference in its entirety.

Specifically, the object of the present disclosure is achieved by the use of one or more dialkyl fumarates of the formula:

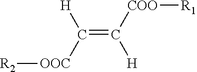

wherein R1 and R2, which may be the same or different, independently represent a linear, branched, or cyclic, saturated or unsaturated C1-20 alkyl radical which may be optionally substituted with halogen (Cl, F, I, Br), hydroxy, alkoxy, nitro, or cyano for preparing a pharmaceutical preparation for use in a composition for the treatment of a heart failure disease.

The C1-20 alkyl radicals, preferably C1-8 alkyl radicals, most preferably C1-5 alkyl radicals are, for example, methyl, ethyl, n-propyl, isopropyl, n-butyl, sec-butyl, t-butyl, pentyl, cyclopentyl, 2-ethyl hexyl, hexyl, cyclohexyl, heptyl, cycloheptyl, octyl, vinyl, allyl, 2-hydroxyethyl, 2 or 3-hydroxy propyl, 2-methoxy ethyl, methoxy methyl, or 2- or 3-methoxy propyl. In one embodiment, at least one of the radicals or R2 is C1-5 alkyl, especially methyl or ethyl. In one embodiment, R1 and R2 are the same or different C1-5 alkyl radicals such as methyl, ethyl, n-propyl, or t-butyl. In one embodiment, R1 and R2 are identical and are methyl or ethyl. In one embodiment, one or more dialkyl fumarates are dimethyl fumarate, methyl ethyl fumarate, and diethyl fumarate.

The dialkyl fumarates to be used according to the present disclosure are prepared by processes known in the art (see, for example, EP 0 312 697, the disclosure of which is incorporated by reference in its entirety).

For example, the active ingredients are used for preparing oral preparations in the form of tablets, micro-tablets, pellets, or granulates, optionally in capsules or sachets. Preparations in the form of micro-tablets or pellets, optionally filled in capsules or sachets are also a subject matter of the present disclosure. The oral preparations may be provided with an enteric coating. Capsules may be soft or hard gelatin capsules.

The dialkyl fumarates used according to the present disclosure may be used alone or as a mixture of several compounds, optionally in combination with the customary carriers and excipients. In some embodiments, the amounts to be used are selected in such a manner that the preparations obtained contain the active ingredient in an amount corresponding to 10 to 300 mg of fumaric acid.

In some embodiments, the preparations according to the present disclosure comprise a total amount of 10 to 300 mg of dimethyl fumarate and/or diethyl fumarate.

In some embodiments, the size or the mean diameter, respectively, of the pellets or micro-tablets is in the range from 300 to 2,000 μm, and in one embodiment, in the range of 500 or 1,000 μm.

In addition to the preparations for peroral administration in the form of micro-pellets, micro-tablets, capsules (such as soft and hard gelatine capsules), granulates, and tablets cited above, suitable pharmaceutical preparations are preparations for cutaneous and transdermal administration in the form of ointments, plasters, lotions, or shower preparations and for parenteral administration in the form of aqueous micro-dispersions, oil-in-water emulsions, or oily solutions for rectal administration of suppositories or micro-enemas. Pharmaceutical preparations in the form of micro-tablets or micro-pellets are preferred for the therapy of a heart failure disease.

According to the present disclosure, administration of dialkyl fumarates may also be carried out in combination with administration of one or more preparations of other heart failure medications, such as but not limited to diuretics, ace-inhibitors, beta-blockers, angiotensin receptor blockers, vasodilators, angiotensin receptor-neprilysin inhibitors, aldosterone antagonists, or combinations thereof. For this purpose, the preparations administered may contain a combination of the active ingredients in the known dosages or amounts, respectively. Likewise, the combination therapy may comprise the parallel administration of separate preparations, by the same or different routes. Optionally, the dosage of the active ingredient contained in addition to the dose of the fumaric acid derivative administered in accordance with the present disclosure may be reduced advantageously.

Dialkyl fumarates, acting on the NRF2/ARE pathway, work on a separate portion of the anti-oxidant cascade compared to other anti-oxidant compounds such as PDE5 inhibitors, statins, ARNIs, aldosterone inhibitors, and antitumor necrosis factor-alpha therapies. Thus, when dialkyl fumarates are used in combination with at least one of these compounds, there happens to be an unexpected synergistic effect beneficial in the treatment of a heart failure disease. According to the present disclosure, administration of dialkyl fumarates may also be carried out in combination with the administration of one or more pharmaceutical preparations with known anti-oxidant properties, such as but not limited to PDE5 inhibitors, statins, angiotensin receptor-neprilysin inhibitors (ARNI), angiotensin receptor blockers (ARB), neprilysin inhibitors, aldosterone inhibitors, antitumor necrosis factor-alpha therapy or combination thereof. For this purpose, the preparations administered may contain a combination of the active ingredients in the known dosages or amounts, respectively. Likewise, the combination therapy may comprise the parallel administration of separate preparations, by the same or different routes. Optionally, the dosage of the active ingredient contained in addition to the dose of the fumaric acid derivative administered in accordance with the present disclosure may be reduced advantageously.

In one embodiment, dimethyl fumarate (DMF) is administered either separately or together with a statin. In another embodiment, a 120 mg or 240 mg dose of DMF is given daily, twice daily (BID), or three times daily (TID) to a patient with a statin dosage between 10 mg to 80 mg. In another embodiment, dimethyl fumarate is administered at a dose of 120 mg to 720 mg and a statin is given at a dose of 10 mg to 80 mg.

In yet another embodiment the statin is selected from group consisting of atorvastatin, fluvastatin, lovastatin, pravastatin, rosuvastatin, and simvastatin.

In one embodiment, dimethyl fumarate (DMF) is administered either separately or together with an angiotensin receptor-neprilysin inhibitor (ARNI) and optionally a statin. In one embodiment, the angiotensin receptor-neprilysin inhibitor (ARNI) is LCZ696 (combination of valsartan and sacubitril) and the statin is selected from group consisting of atorvastatin, fluvastatin, lovastatin, pravastatin, rosuvastatin, and simvastatin. In yet another embodiment, a 120 mg or 240 mg dose of DMF is given daily, BID, or TID, to a patient with LCZ696 50 mg to 400 mg daily and optionally a statin dosage between 10 mg to 80 mg.

By administration of the dialkyl fumarates in the form of micro-tablets, in one embodiment, gastrointestinal irritations and side effects, which are reduced already when conventional tablets are administered but is still observed, may be further reduced when using fumaric acid derivatives and salts.

It is presumed that, upon administration of conventional tablets, the ingredients of the tablet are released in the intestine in a concentration which is too high, causing local irritation of the intestinal mucous membrane. This local irritation results in a short-term release of very high TNF-α concentrations, which may be responsible for the gastrointestinal side effects. In the case of enteric-coated micro-tablets in capsules, very low local concentrations of the active ingredients in the intestinal epithelial cells are achieved. The micro-tablets are incrementally released by the stomach and passed into the small intestine by peristaltic movements so that distribution of the active ingredients is improved.

This means that enteric-coated micro-tablets in the same dosage are distributed already in the stomach and passed to the intestine in portions where the active ingredients are released in smaller dosages. This avoids local irritation of the intestinal epithelial cells and the release of TNF-α. It is assumed that this results in the improved tolerance of micro-tablets in the gastrointestinal tract versus conventional tablets.

In addition, resorption is improved, because the dialkyl fumarates to be used according to the present disclosure are not the active ingredient per se, but a so-called prodrug, which must be converted into the active ingredient in the body.

### Pharmaceutical Preparation Example

A pharmaceutical composition is formulated to be compatible with its intended route of administration. Methods to accomplish the administration are known in the art.

In principle, the oral preparations according to the present disclosure are in the form of tablets or micro-tablets prepared by classical tabletting processes. Alternatively, other methods for the preparation of tablets may be used, such as direct tabletting and processes for preparing solid dispersions in according with the melt method and the spray drying method. The tablets may be provided with an enteric coating. The enteric coating may be applied in a classical coating pan or sprayed on or applied in a fluidised-bed apparatus. The tablet may also be provided with a film coat. Examples of some of formulations containing DMF are given in U.S. Pat. No. 6,509,376, the disclosure of which is herein incorporated by reference in its entirety.

### Prophetic Example 1

The following prophetic example serves to provide approximate dosage levels of dimethyl fumarate to achieve the intended effect, for example treatment of chronic heart failure with preserved ejection fraction (HFPEF). Based on the literature, a few assumptions about the dosage can be made, as will be described in further detail below.

The full mechanism of fumaric acid esters such as dimethyl fumarate (DMF) and its primary metabolite, monomethyl fumarate (MMF), is not completely understood, but their beneficial effects appear to be mediated, at least in part, through the activation of the NRF2 antioxidant response pathway, which further increases expression of ARE, which increases expression of detoxifying enzymes and antioxidant proteins.

NRF2 deficiency, demonstrated by NRF2 knockout in murine models, results in an earlier onset of cardiac dysfunction induced by pressure and volume overload (Li et al Arterioscler Thromb Vasc Biol. 2009, 29(11), 1843-50). Certain NRF2 activators such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α-estradiol have been used as therapeutic targets to reduce cardiac remodeling, but dimethyl fumarate have not been used yet to reduce cardiac remodeling (Zhou et al; J Appl Physiol. 2015, 119(8), 944-951).

Fumarates are cardioprotective in acute situations via activation of the NRF2 pathway in acute ischemia due to myocardial infarction (Ashrafian et. al; Cell Metab. 2012, 15(3), 361-71). However, Ashrafian et. al claims that fumarates are harmful in chronic situations, including heart failure.

Dimethyl Fumarate has been tested for multiple sclerosis and psoriasis at multiple dosages in the past, including 120 mg, 240 mg, daily, BID, and TID. The side effect profile was similar regardless of which dosage was used. In order to determine dosage of DMF for HFPEF, various levels of DMF (120 mg, 240 mg, daily, BID, and TID) will be administered to patients. In one embodiment, dimethyl fumarate is administered at a dose of 120 mg to 720 mg.

Dimethyl fumarate has never been tested chronically in humans to see long term cardiac effects, for example, for chronic treatment of HFPEF. A daily dose of dimethyl fumarate will be administered over the long term or over a prolonged period of time (e.g., one month, two months, three months, four months, five months, six months, seven months, eight months, nine months, ten months, eleven months, twelve months, one year, two years, three years, four years, five years, ten years, fifteen years, twenty years, etc.). In some embodiments, long term administration may include a drug holiday, for example, daily dose given for one month, two weeks off, one month on etc.

Furthermore, pro-inflammatory cytokines IL-6 and TNF-α are raised in HFPEF, which may lead to increase activity of VCAM, E-Selection, and NADPH oxidase, which increase ROS in coronary microvasculature endothelial cells, leading to the hallmarks of HFPEF: ventricular stiffness, impaired relaxation, and cardiac dysfunction. The prodrugs of monomethyl fumarate may reduce damage of ROS in heart failure by multiple pathways including increasing the NRF2/ARE pathway, and possibly by reducing NF-kB, which reduces IL-6 and TNF-α.

LCZ696, a combined angiotensin receptor neprilysin inhibitor (ARNI), has recently shown to reduce mortality in HFREF but not yet in HFPEF patients. LCZ696 inhibits natriuretic peptide breakdown and enhances cGMP activation, and in HFPEF was associated with incremental reductions in circulating N-terminal pro-B-type natriuretic peptide (NT-proBNP) levels when compared to treatment with the ARB valsartan, alone. However, these reductions were incremental, and it is yet to be seen whether LCZ696 or other angiotensin receptor-neprilysin inhibitors will lead to any significant mortality or clinical benefit in HFPEF patients. Furthermore, the comparison with ARB valsartan alone is flawed in that ARB valsartan is used in the treatment of HFREF but not in HFPEF.

The patient's baseline TNF-alpha, IL-6, NT-proBNP will be measured at the start of the trial and compared to levels at various intervals (weeks to months to years) to determine the ideal dosage based on reductions in TNF-alpha, IL-6, and/or NT-proBNP as a quantitative endpoint. Other endpoints will be compared as well, such as NYHA Heart Failure class, hospital visits and admissions, and need for other heart failure medications. Once a proposed dosage is chosen (120 mg vs 240 mg, daily, BID, or TID), such a dosage will then be tested in a larger group of HFPEF patients to measure changes in morbidity and mortality.

### Prophetic Example 2

Based on the above prophetic example, an exemplary, non-limiting embodiment is described in detail below. As described herein, a user may include a male or female between the ages of 50 to 100 with ejection fraction of greater than 40%, and more likely to be a female with a documented history of high blood pressure, diabetes, and/or COPD; with at least one episode of fluid overload; or who has HFPEF or is at risk of developing (HFPEF).

The most common disease leading to HFPEF is systolic hypertension, which is present in more than 85% of patients. Patients with HFPEF have normal LV end-diastolic volume and normal (or near-normal) EF and stroke volume and commonly exhibit concentric remodeling of either the LV chamber and/or cardiomyocytes. Patients with HFPEF have a devastating 5-year mortality rate (approaching 60%), costly morbidity (6-month hospitalization rate of 50%), and debilitating symptoms (maximum myocardial oxygen consumption [MVO2] averaging 14 mL/g/min). In some embodiments, as described herein, treating heart failure includes reducing mortality due to heart failure. In some embodiments, treating heart failure includes increasing a maximum myocardial oxygen consumption in a subject experiencing heart failure.

More than half of heart failure patients have heart failure with preserved ejection fraction (HFPEF). Morbidity and mortality of HFPEF are similar to traditional HF; however, medications proven effective in HFREF have not been found to be effective in HFPEF. At present there are no approved treatments to reduce mortality in HFPEF. In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in patients with HFPEF, and are not approved therapies for HFPEF.

### Prophetic Example 3

The following prophetic example serves to provide a combination therapy for patients with HFPEF, which includes DMF with a statin. To date, there has been no prospective study of statins in patients with HFPEF. However, statins have pleotropic effects, in which they have been shown to be beneficial to non-HFPEF patients beyond what is predicted based on their ability to reduce cholesterol, likely through anti-inflammatory pathways. By combining a statin with a DMF, we expect a synergistic effect to reduce the reactive oxygen species associated with HFPEF, which in turn will reduce stiffness in HFPEF and also reduce biomarkers such as IL-6, TNF-alpha, or NT-proBNP, ultimately improving cardiac remodeling and survival in HFPEF patients. In one such example, a 120 mg or 240 mg dose of DMF is given daily, BID, or TID to a patient with a statin dosage between 10 mg to 80 mg. In another embodiment, dimethyl fumarate is administered at a dose of 120 mg to 720 mg and a statin at a dose of 10 mg to 80 mg.

### Section II: Fatty-Acid Fumarate Derivatives

The FAFDs possess the ability to treat or prevent heart failure diseases, including heart failure with preserved ejection fraction (HFPEF).

The FAFDs have been designed to bring together fumaric acid and ester analogs thereof and fatty acids into a single molecular conjugate. The activity of the FAFDs is substantially greater than the sum of the components suggesting that the activity induced by the FAFDs is synergistic.

### Definitions

The following definitions are used in connection with the FAFDs.

The term “fatty acid fumarate derivatives” of “FAFD” includes any and all possible isomers, stereoisomers, enantiomers, diastereomers, tautomers, pharmaceutically acceptable salts, hydrates, solvates, and prodrugs of the FAFDs described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl) or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C1-C4 alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C1-C5 alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a monocyclic or bicyclic hydrocarbon containing 3-12 carbon atoms wherein at least one of the carbon atoms is substituted with a O, N, or S. Examples of a heterocycle include, but are not limited to, aziridine, oxirane, thiirane, azetidine, oxetane, thietane, pyrrolidine, tetrahydrofurane, tetrahydrothiophene, piperidine, tetrahydropyran, thiane, imidazolidine, oxazolidine, thiazolidine, dioxolane, dithiolane, piperazine, oxazine, dithiane, dioxane, diazabicycloheptane and diazabicyclooctane.

The term “heteroaryl” as used herein refers to a monocyclic or bicyclic ring structure having 5 to 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid, fatty acids that are metabolized in vivo to omega-3 fatty acids, and lipoic acid. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), tetracosahexaenoic acid (nisinic acid or all-cis-6,9,12,15,18,21-tetracosenoic acid) and stereoisomers of lipoic acid.

A “subject” is a mammal, e.g., a human, mouse, rat, guinea pig, dog, cat, horse, cow, pig, or non-human primate, such as a monkey, chimpanzee, baboon or rhesus.

The invention also includes pharmaceutical compositions comprising an effective amount of a FAFD and a pharmaceutically acceptable carrier. The invention includes a FAFD when provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “heart failure disease” may be heart failure with preserved ejection fraction (HFPEF); heart failure with ejection fraction ≥40%; diastolic heart failure; heart failure with elevated levels of TNF-α, IL-6, CRP, or TGF-β; hypertension with a risk of developing HFPEF; atrial fibrillation with a risk of developing HFPEF; diabetes with a risk of developing HFPEF; COPD with a risk of developing HFPEF; ischemic heart disease with a risk of developing HFPEF; obesity with a risk of developing HFPEF; chronic heart failure; compensated heart failure; decompensated heart failure; or other conditions known to have a high risk of developing HFPEF. In particular, heart failure disease is heart failure with preserved ejection fraction (HFPEF).

An “effective amount” when used in connection with a FAFD is an amount effective for treating or preventing a metabolic disorder.

The term “carrier,” as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating,” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer,” “administering,” or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound, which is convertible in vivo by metabolic means (e.g., by hydrolysis) to a FAFD.

### Compounds

Certain embodiments of methods disclosed herein use a FAFD according to Formulas I, IA, IB, IC, and II, as set forth below.

Certain embodiments of a method disclosed herein use a compound of Formula I and Formula II:

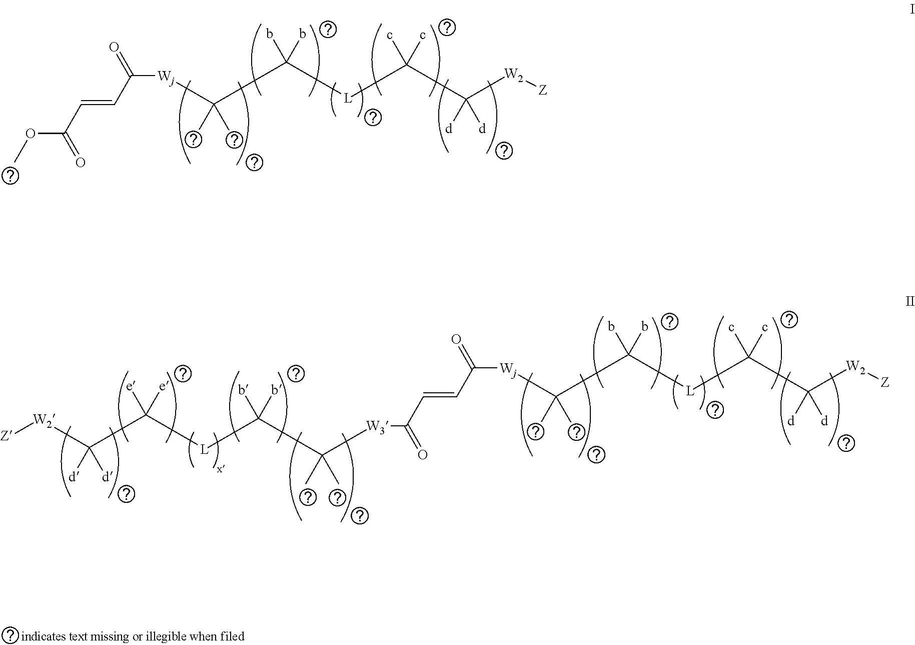

and pharmaceutically acceptable salts, hydrates, solvates, prodrugs, enantiomers, and stereoisomers thereof;

wherein

W1, W2, a, b, c, d, m, ml, n, o, p, q, L, Z, r, s, t, v, R, R1, R2, R3, R4, R5, R6, W1′, W2′, a′, c′, b′, d′, n′, o′, p′, q′, ml′, L′, and Z′ is as defined above for Formula I and Formula II, provided that there is at least one of the following in the compound.

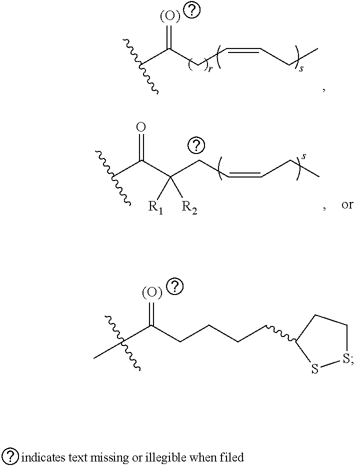

In some embodiments, one Z is

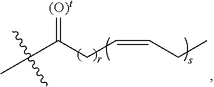

and r is 2.

In some embodiments, one Z is

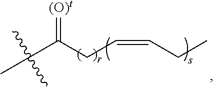

and r is 3.

In some embodiments, one Z is

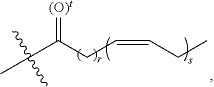

and r is 7.

In other embodiments, one Z is

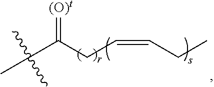

and s is 3.

In some embodiments, one Z is

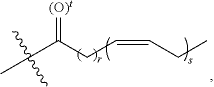

and s is 5.

In some embodiments, one Z is

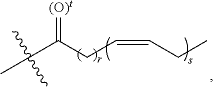

and s is 6.

In some embodiments, one Z is

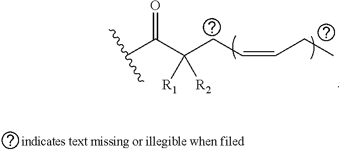

and v is 1.

In other embodiments, one Z is

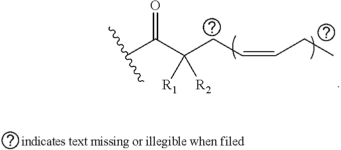

and v is 2.

In some embodiments, one Z is

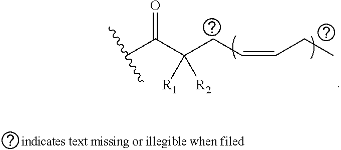

and v is 6.

In some embodiments, one Z is

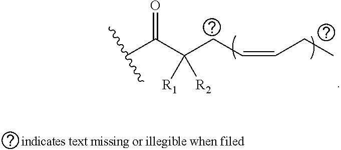

and s is 3.

In some embodiments, one Z is

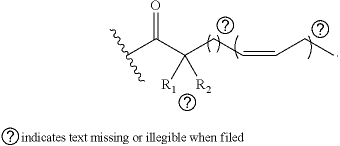

and s is 5.

In other embodiments, one Z is

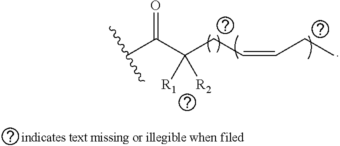

and s is 6.

In other embodiments, Z is

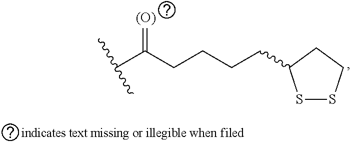

and t is 1.

In some embodiments, Z is

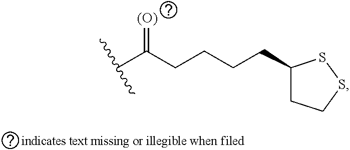

and t is 1.

In another aspect of methods disclosed herein use any compounds of Formula IA:

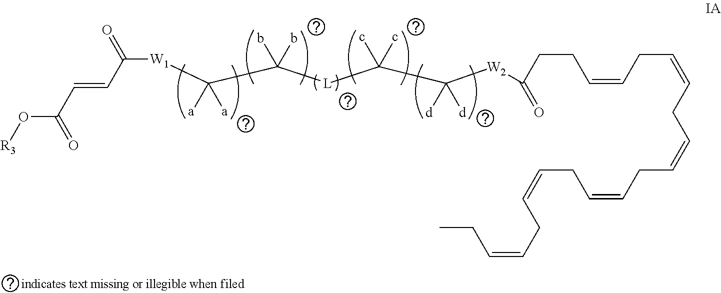

and pharmaceutically acceptable salts, hydrates, solvates, prodrugs, enantiomers, and stereoisomers thereof;

wherein

W1, W2, a, b, c, d, m, ml, n, o, p, q, L, R, R1, R2, R3, R4, R5, R6 are as defined above for Formula IA.

Certain embodiments of methods disclosed herein use a compound of Formula IB:

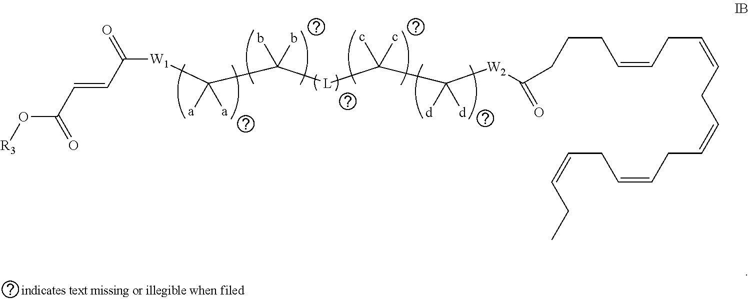

and pharmaceutically acceptable salts, hydrates, solvates, prodrugs, enantiomers, and stereoisomers thereof;

wherein

W1, W2, a, b, c, d, m, ml, n, o, p, q, L, R, R1, R2, R3, R4, R5, R6 are as defined above for Formula IB.

Certain embodiments of methods disclosed herein use a compound of Formula IC:

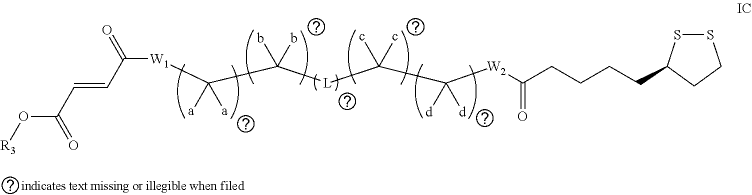

and pharmaceutically acceptable salts, hydrates, solvates, prodrugs, enantiomers, and stereoisomers thereof;

wherein

W1, W2, a, b, c, d, m, ml, n, o, p, q, L, R, R1, R2, R3, R4, R5, and R6 are as defined above for Formula IC.

The following embodiments are illustrative of a method of using compounds of Formulas I, IA, IB, IC, and II.

In some embodiments of a method of using compounds of Formula I, IA, IB, IC, and II, R3 is CH3.

In some embodiments of a method of using compounds of Formula I, IA, IB, IC, and II, R3 is —CH2CH3.

In some embodiments of a method of using compounds of Formula I, IA, IB, IC, and II, R3 is H.

In some embodiments of a method of using compounds of Formula I, IA, IB, IC, and II, W1 is NH.

In some embodiments of a method of using compounds of Formula I, IA, IB, IC, and II, W2 is NH.

In some embodiments of a method of using compounds of Formula I, IA, IB, IC, and II, Wt is O.

In some embodiments of a method of using compounds of Formula I, IA, IB, IC, and II, W2 is O.

In some embodiments of a method of using compounds of Formula I, IA, IB, IC, and II, W1 is null.

In some embodiments of a method of using compounds of Formula I, IA, IB, IC, and II, W2 is null.

In some embodiments of a method of using compounds of Formula I, IA, IB, IC, and II, W1 and W2 are each NH.

In some embodiments of a method of using compounds of Formula I, IA, IB, IC, and II, W1 and W2 are each null.

In some embodiments of a method of using compounds of Formula I, IA, IB, IC, and II, W1 is O and W2 are NH.

In some embodiments of a method of using compounds of Formula I, IA, IB, IC, and II, W1 and W2 are NR, and R is CH3.

In some embodiments of a method of using compounds of Formula I, IA, IB, IC, and II, m is 0.

In other embodiments of a method of using compounds of Formula I, IA, IB, IC, and II, m is 1.

In other embodiments of a method of using compounds of Formula I, IA, IB, IC, and II, m is 2.

In some embodiments of a method of using compounds of Formula I, IA, IB, IC, and II, L is —S— or —S—S—.

In some embodiments of a method of using compounds of Formula I, IA, IB, IC, and II, L is —O—.

In some embodiments of a method of using compounds of Formula I, IA, IB, IC, and II, L is —C(O)—.

In some embodiments of a method of using compounds of Formula I, IA, IB, IC, and II, L is heteroaryl.

In some embodiments of a method of using compounds of Formula I, IA, IB, IC, and II, L is heterocycle.

In some embodiments of a method of using compounds of Formula I, IA, IB, IC, and II, L is

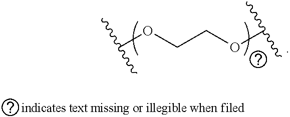

In some embodiments of a method of using compounds of Formula I, IA, IB, IC, and II, L is

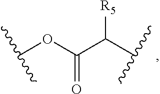

In some embodiments of a method of using compounds of Formula I, IA, IB, IC, and II, L is

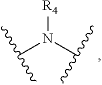

In some embodiments of a method of using compounds of Formula I, IA, IB, IC, and II, L is

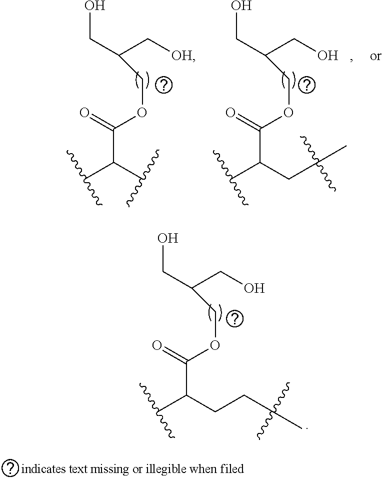

In some embodiments of a method of using compounds of Formula I, IA, IB, IC, and II, L is

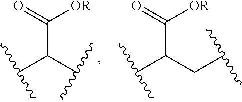

In some embodiments of a method of using compounds of Formula I, IA, IB, IC, and II, L is

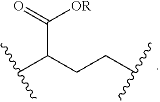

In some embodiments of a method of using compounds of Formula I, IA, IB, IC, and II, L is

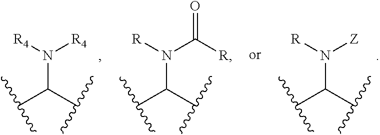

In some embodiments of a method of using compounds of Formula I, IA, IB, IC, and II, L is

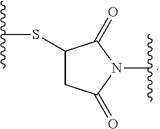 [SMILES: *SC1CC(=O)N(*)C1=O]

In some embodiments of a method of using compounds of Formula I, IA, IB, IC, and II, L is

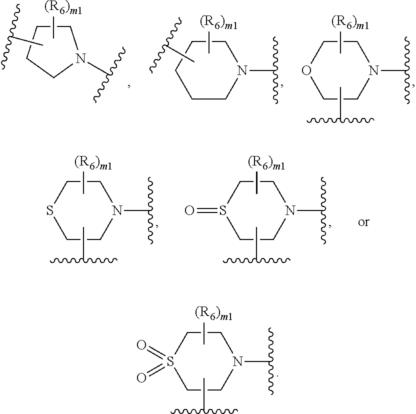

In some embodiments of a method of using compounds of Formula I, IA, IB, IC, and II, L is

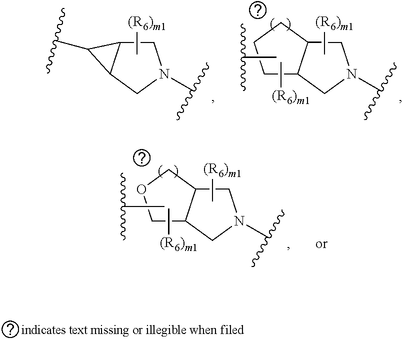

In some embodiments of a method of using compounds of Formula I, IA, IB, IC, and II, L is

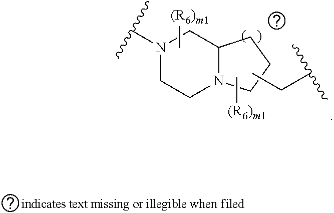

In some embodiments of a method of using compounds of Formula I, IA, IB, IC, and II, L is

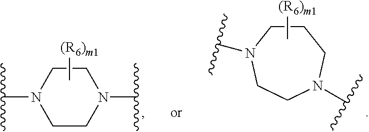

In some embodiments of a method of using compounds of Formula I, IA, IB, IC, and II, L is

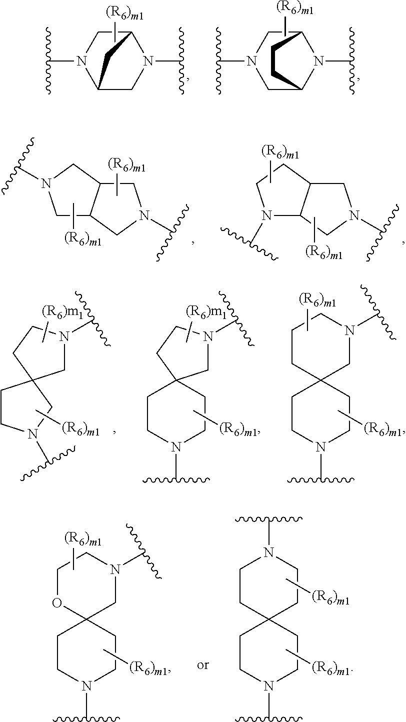

In other embodiments of a method of using compounds of Formula I, IA, IB, IC, and II, one of n, o, p, and q is 1.

In some embodiments of a method of using compounds of Formula I, IA, IB, IC, and II, two of n, o, p, and q are each 1.

In other embodiments of a method of using compounds of Formula I, IA, IB, IC, and II, three of n, o, p, and q are each 1.

In some embodiments of a method of using compounds of Formula I, IA, IB, IC, and II, n, o, p, and q are each 1.

In some embodiments of a method of using compounds of Formula I, IA, IB, IC, and II, two of n, o, p, and q are each 1 and the other two are each 0.

In some embodiments of a method of using compounds of Formula I, IA, IB, IC, and II, r is 2 and s is 6.

In some embodiments of a method of using compounds of Formula I, IA, IB, IC, and II, r is 3 and s is 5.

In some embodiments of a method of using compounds of Formula I, IA, IB, IC, and II, t is 1.

In some embodiments of a method of using compounds of Formula I, IA, IB, IC, and II, W1 and W2 are each NH; m is 0; n and o are each 1; and p and q are each 0.

In some embodiments of a method of using compounds of Formula I, IA, IB, IC, and II, W1 and W2 are each NH; m is 1; n, o, p, and q are each 1; and L is O.

In some embodiments of a method of using compounds of Formula I, IA, IB, IC, and II, W1 and W2 are each NH; m is 1; n, o, p, and q are each 1; and L is

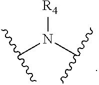

In some embodiments of a method of using compounds of Formula I, IA, IB, IC, and II, W1 and W2 are each NH; m is 1; n, o, p, and q are each 1; and L is —S—S—.

In some embodiments of a method of using compounds of Formula I, IA, IB, IC, and II, W1 and W2 are each NH; m is 1; n and o are each 0; p and q are each 1; and L is

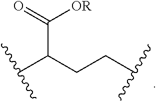

In some embodiments of a method of using compounds of Formula I, IA, IB, IC, and II, W1 and W2 are each NH; m is 1; k is O; n and o are each 0; p and q are each 1; and L is

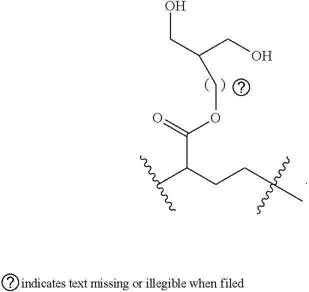

In some embodiments of a method of using compounds of Formula I, IA, IB, IC, and II, W1 and W2 are each NH; m is 1; n and o are each 1; p and q are each 0; and L is

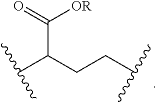

In some embodiments of a method of using compounds of Formula I, IA, IB, IC, and II, W1 and W2 are each NH; m is 1; k is 0; n is 1; o, p and q are each 0; and L is

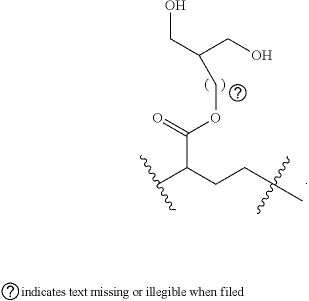

In some embodiments of a method of using compounds of Formula I, IA, IB, IC, and II, W1 and W2 are each NH; m is 1; n, o, and p are each 0; and q is 1; and L is

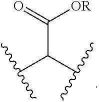

In some embodiments of a method of using compounds of Formula I, IA, IB, IC, and II, W1 and W2 are each NH; m is 1; k is 1; n, o, and p are each 0; and q is 1; and L is

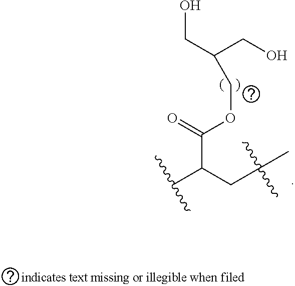

In some embodiments of a method of using compounds of Formula I, IA, IB, IC, and II, W₁ and W₂ are each NH; m is 1; n is 1; and o, p, and q are each 0; and L is

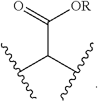

In some embodiments of a method of using compounds of Formula I, IA, IB, IC, and II, W1 and W2 are each NH; m is 1; k is 1; o, p, and q are each 0; and L is

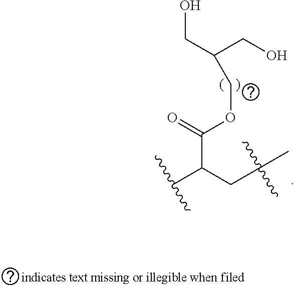

In some embodiments of a method of using compounds of Formula I, IA, IB, IC, and II, W1 and W2 are each NH; m is 1; n, o, p, and q are each 1; and L is

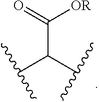

In some embodiments of a method of using compounds of Formula I, IA, IB, IC, and II, W₁ and W₂ are each NH; m is 1; n, o, p, and q are each 1; and L is

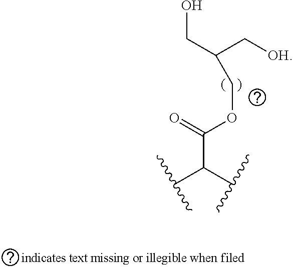

In some embodiments of a method of using compounds of Formula I, IA, IB, IC, and II, W1 and W2 are each NH; m is 0; k is 1; o and p are each 1; and q is O.

In some embodiments of a method of using compounds of Formula I, IA, IB, IC, and II, W1 and W2 are each NH; m is 0; and n, o, p, and q are each 1.

In some embodiments of a method of using compounds of Formula I, IA, IB, IC, and II, W1 and W2 are each NH; m is 0; n and o are each 1; p and q are each 0; and each a is CH3.

In some embodiments of a method of using compounds of Formula I, IA, IB, IC, and II, W1 and W2 are each NH; m is 0; n and o are each 1; p and q are each 0; and each b is CH3.

In some embodiments of a method of using compounds of Formula I, IA, IB, IC, and II, W1 and W2 are each NH; m is 1; n, o, p, and q are each 1; R4 is H; and L is

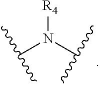

In some embodiments of a method of using compounds of Formula I, IA, IB, IC, and II, W₁ and W2 are each NH; m is 1; n, p and q are each 1; and o is 2; R4 is H; and L is

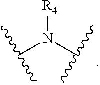

In some embodiments of a method of using compounds of Formula I, IA, IB, IC, and II, W₁ and W₂ are each NH; m is 1; n, o, p are each 1; and q is 2; and L is

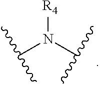

In some embodiments of a method of using compounds of Formula I, IA, IB, IC, and II, W1 and W2 are each NH; m is 1; n, o, p, and q are each 1; and L is

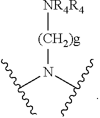

In some embodiments of a method of using compounds of Formula I, IA, IB, IC, and II, W1 and W2 are each NH; m is 1; n and p are each 1; and o and q are each 0; and L is —C(O)—.

In some embodiments of a method of using compounds of Formula I, IA, IB, IC, and II, W1 and W2 are each NH; m is 1; n and p are each 1; and o and q are each 0; and L is

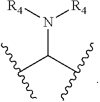

In some embodiments of a method of using compounds of Formula I, IA, IB, IC, and II, W₁ and W₂ are each NH; m is 1; n, o, p, q are each 1; and L is

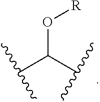

In some embodiments of a method of using compounds of Formula I, IA, IB, IC, and II, W₁ and W₂ are each NH; m is 1; n, o, p, and q are each 1; h is 1; and L is

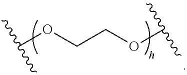

In some embodiments of a method of using compounds of Formula I, IA, IB, IC, and II, W1 and W2 are each NH; m is 1; n, o, p, and q are each 1; and L is —S—.

In some embodiments of a method of using compounds of Formula I, IA, IB, IC, and II, W1 and W2 are each NH; m is 1; n, o, p are each 0; q is 1; one d is —CH3; and L is

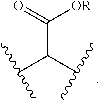

In some embodiments of a method of using compounds of Formula I, IA, IB, IC, and II, W1 and W2 are each NH; m is 2; n, o, p, and q are each 0; one L is

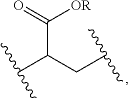

and one L is

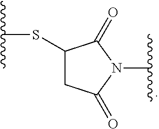 [SMILES: *SC1CC(=O)N(*)C1=O]

In some embodiments of a method of using compounds of Formula I, IA, IB, IC, and II, m is 0; n, o, p, and q are each 0; and W1 and W2 are taken together to form an optionally substituted piperazine group.

In some embodiments of a method of using compounds of Formula I, IA, IB, IC, and II, m is 1; n, o, p, and q are each 0; W1 and W2 are each null; and L is

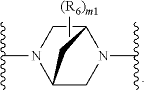

In some embodiments of a method of using compounds of Formula I, IA, IB, IC, and II, m is 1; n and p are each 1; o and q are each 0; W1 and W2 are each NH; and L is C3-C6 cycloalkyl.

In some embodiments of a method of using compounds of Formula I, IA, IB, IC, and II, m is 1; n is 1; o, p, and q are each 0; W1 and W2 are each NH, and L is C3-C6 cycloalkyl.

In some embodiments of a method of using compounds of Formula I, IA, IB, IC, and II, m is 1; n, o, p, are each 0; q is 1; W1 and W2 are each NH, and L is C3-C6 cycloalkyl.

In some embodiments of a method of using compounds of Formula I, IA, IB, IC, and II, m is 1; n, o, p, and q are each 0; W1 is NH; W2 is null; and L is

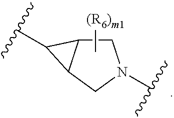

In some embodiments of a method of using compounds of Formula I, IA, IB, IC, and II, m is 1; n, o, p, and q are each 0; W1 is null; W2 is NH; and L is

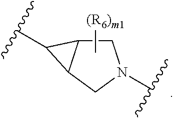

In some embodiments of a method of using compounds of Formula I, IA, IB, IC, and II, m is 1; n, o, p, and q are each 0; W1 is NH; W2 is null; and L is

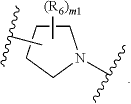

In some embodiments of a method of using compounds of Formula I, IA, IB, IC, and II, m is 1; n, o, p, and q are each 0; W1 is null; W2 is NH; and L is

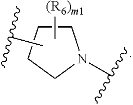

In some embodiments of method using compounds of Formula I, IA, IB, IC, and II, m is 1; n is 1; o, p, and q are each 0; W1 is NH; W2 is null; and L is

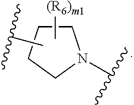

In some embodiments of a method of using compounds of Formula I, IA, IB, IC, and II, m is 1; n, o, p, are each 0; q is 1; W1 is null; W2 is NH; and L is

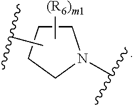

In some embodiments of a method of using compounds of Formula I, IA, IB, IC, and II, m is 1; n, o, p, and q are each 0; W1 is NH; W2 is null; and L is

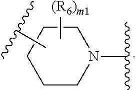

In some embodiments of a method of using compounds of Formula I, IA, IB, IC, and II, m is 1; n, o, p, and q are each 0; W1 is null; W2 is NH; and L is

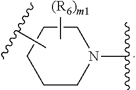

In some embodiments of a method of using compounds of Formula I, IA, IB, IC, and II, m is 1; n is 1; o, p, and q are each 0; W1 is NH; W2 is null; and L is

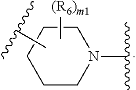

In some embodiments of a method of using compounds of Formula I, IA, IB, IC, and II, m is 1; n, o, p, are each 0; q is 1; W1 is null; W2 is NH; and L is

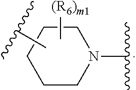

In some embodiments of a method of using compounds of Formula I, IA, IB, IC, and II, m is 1; n is 1; o, p, and q are each 0; W1 is NH; W2 is null; and L is

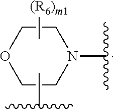

In some embodiments of a method of using compounds of Formula I, IA, IB, IC, and II, m is 1; n, o, p, are each 0; q is 1; W1 is null; W2 is NH; and L is

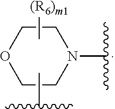

In some embodiments of a method of using compounds of Formula I, IA, IB, IC, and II, m is 1; n, o, p, q are each 0; W1 and W2 are null; and L is

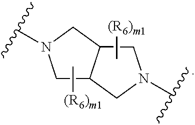

In some embodiments of a method of using compounds of Formula I, IA, IB, IC, and II, m is 1; n, o, p, q are each 0; W1 and W2 are null; and L is

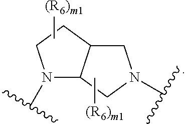

In some embodiments of a method of using compounds of Formula I, IA, IB, IC, and II, m is 1; n, o, p, q are each 0; W1 is NH; W2 is null; and L is

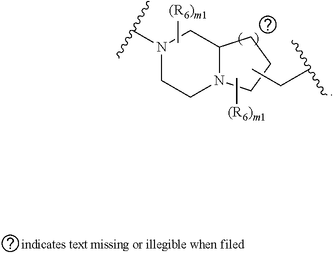

In some embodiments of a method of using compounds of Formula I, IA, IB, IC, and II, m is 1; n, o, p, q are each 0; W1 is null; W2 is NH; and L is

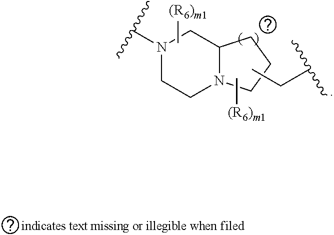

In some embodiments of a method of using compounds of Formula I, IA, IB, IC, and II, m is 1; n, o, p, are each 0; q is 1; W1 and W2 are each NH; and L is

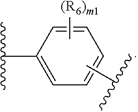

In some embodiments of a method of using compounds of Formula I, IA, IB, IC, and II, m is 1; n, o, p, are each 0; q is 1; W1 and W2 are each NH; and L is a heteroaryl.

In some of the foregoing embodiments of a method of using compounds of Formula I, IA, IB, IC, and II, r is 2; s is 6; and t is 1.

In some of the foregoing embodiments of a method of using compounds of Formula I, IA, IB, IC, and II, r is 3; s is 5; and t is 1.

In some of the foregoing embodiments of a method of using compounds of Formula I, IA, IB, IC, and II, Z is

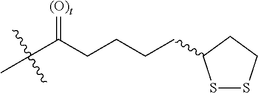

and t is 1.

In the method of using compounds of Formula I, IA, IB, IC and II, any one or more of H may be substituted with a deuterium.

In other illustrative embodiments, a method of using compounds of Formula I, IA, IB, IC and II are as set forth below:
(E)-methyl 4-(2-(4Z,7Z,10Z,13Z,16Z,19Z)-docosa-4,7,10,13,16,19-hexaenamidoethylamino)-4-oxobut-2-enoate (I-1);
(E)-methyl 4-(2-(5Z,8Z,11Z,14Z,17Z)-icosa-5,8,11,14,17-pentaenamidoethylamino)-4-oxobut-2-enoate (I-2);
(E)-methyl 4-(2-(2-(4Z,7Z,10Z,13Z,16Z,19Z)-docosa-4,7,10,13,16,19-hexaenamidoethoxy)ethylamino)-4-oxobut-2-enoate (I-3);
(E)-methyl 4-(2-((2-(4Z,7Z,10Z,13Z,16Z,19Z)-docosa-4,7,10,13,16,19-hexaenamidoethyl)(methyl)amino)ethylamino)-4-oxobut-2-enoate (I-4);
(E)-methyl 4-(2-(2-(2-(4Z,7Z,10Z,13Z,16Z,19Z)-docosa-4,7,10,13,16,19-hexaenamidoethyl)disulfanyl)ethylamino)-4-oxobut-2-enoate (I-5);
(S)-methyl 6-((4Z,7Z,10Z,13Z,16Z,19Z)-docosa-4,7,10,13,16,19-hexaenamido)-2-((E)-4-methoxy-4-oxobut-2-enamido)hexanoate (I-6);
(S)-6-((4Z,7Z,10Z,13Z,16Z,19Z)-docosa-4,7,10,13,16,19-hexaenamido)-2-((E)-4-methoxy-4-oxobut-2-enamido)hexanoic acid (I-7);
(S)-1,3-dihydroxypropan-2-yl 6-((4Z,7Z,10Z,13Z,16Z,19Z)-docosa-4,7,10,13,16,19-hexaenamido)-2-((E)-4-methoxy-4-oxobut-2-enamido)hexanoate (I-8);
(S)-methyl 2-((4Z,7Z,10Z,13Z,16Z,19Z)-docosa-4,7,10,13,16,19-hexaenamido)-6-((E)-4-methoxy-4-oxobut-2-enamido)hexanoate (I-9);
(S)-2-((4Z,7Z,10Z,13Z,16Z,19Z)-docosa-4,7,10,13,16,19-hexaenamido)-6-((E)-4-methoxy-4-oxobut-2-enamido)hexanoic acid (I-10);
(S)-1,3-dihydroxypropan-2-yl 2-((4Z,7Z,10Z,13Z,16Z,19Z)-docosa-4,7,10,13,16,19-hexaenamido)-6-((E)-4-methoxy-4-oxobut-2-enamido)hexanoate (I-11);
(E)-methyl 4-(3-(4Z,7Z,10Z,13Z,16Z,19Z)-docosa-4,7,10,13,16,19-hexaenamido-1-methoxy-1-oxopropan-2-ylamino)-4-oxobut-2-enoate (I-12);
3-(4Z,7Z,10Z,13Z,16Z,19Z)-docosa-4,7,10,13,16,19-hexaenamido-2-((E)-4-methoxy-4-oxobut-2-enamido)propanoic acid (I-13);
(E)-methyl 4-(1-(1,3-dihydroxypropan-2-yloxy)-3-(4Z,7Z,10Z,13Z,16Z,19Z)-docosa-4,7,10,13,16,19-hexaenamido-1-oxopropan-2-ylamino)-4-oxobut-2-enoate (I-14);
(E)-methyl 4-(2-(4Z,7Z,10Z,13Z,16Z,19Z)-docosa-4,7,10,13,16,19-hexaenamido-3-methoxy-3-oxopropylamino)-4-oxobut-2-enoate (I-15);
2-(4Z,7Z,10Z,13Z,16Z,19Z)-docosa-4,7,10,13,16,19-hexaenamido-3-((E)-4-methoxy-4-oxobut-2-enamido)propanoic acid (I-16);
(E)-methyl 4-(3-(1,3-dihydroxypropan-2-yloxy)-2-(4Z,7Z,10Z,13Z,16Z,19Z)-docosa-4,7,10,13,16,19-hexaenamido-3-oxopropylamino)-4-oxobut-2-enoate (I-17);
2-(2-(4Z,7Z,10Z,13Z,16Z,19Z)-docosa-4,7,10,13,16,19-hexaenamidoethyl)-4-((E)-4-methoxy-4-oxobut-2-enamido)butanoic acid (I-18);
(E)-methyl 4-(3-((1,3-dihydroxypropan-2-yloxy)carbonyl)-5-(4Z,7Z,10Z,13Z,16Z,19Z)-docosa-4,7,10,13,16,19-hexaenamidopentylamino)-4-oxobut-2-enoate (I-19);
(E)-methyl 4-(3-(4Z,7Z,10Z,13Z,16Z,19Z)-docosa-4,7,10,13,16,19-hexaenamidopropylamino)-4-oxobut-2-enoate (I-20);
(E)-methyl 4-(4-(4Z,7Z,10Z,13Z,16Z,19Z)-docosa-4,7,10,13,16,19-hexaenamidobutylamino)-4-oxobut-2-enoate (I-21);
(E)-methyl 4-(1-(4Z,7Z,10Z,13Z,16Z,19Z)-docosa-4,7,10,13,16,19-hexaenamido-2-methylpropan-2-ylamino)-4-oxobut-2-enoate (I-22);
(E)-methyl 4-(2-(4Z,7Z,10Z,13Z,16Z,19Z)-docosa-4,7,10,13,16,19-hexaenamido-2-methylpropylamino)-4-oxobut-2-enoate (I-23);
(E)-methyl 4-(2-(2-(4Z,7Z,10Z,13Z,16Z,19Z)-docosa-4,7,10,13,16,19-hexaenamidoethylamino)ethylamino)-4-oxobut-2-enoate (I-24);
(E)-methyl 4-(3-(2-(4Z,7Z,10Z,13Z,16Z,19Z)-docosa-4,7,10,13,16,19-hexaenamidoethylamino)propylamino)-4-oxobut-2-enoate (I-25);
(E)-methyl 4-(2-(3-(4Z,7Z,10Z,13Z,16Z,19Z)-docosa-4,7,10,13,16,19-hexaenamidopropylamino)ethylamino)-4-oxobut-2-enoate (I-26);
(E)-methyl 4-(2-((3-(4Z,7Z,10Z,13Z,16Z,19Z)-docosa-4,7,10,13,16,19-hexaenamidopropyl)(ethyl)amino)ethylamino)-4-oxobut-2-enoate (I-27);
(E)-methyl 4-(2-(N-(3-(4Z,7Z,100Z,13Z,16Z,19Z)-docosa-4,7,10,13,16,19-hexaenamidopropyl)acetamido)ethylamino)-4-oxobut-2-enoate (I-28);
(E)-methyl 4-(2-((2-(4Z,7Z,10Z,13Z,16Z,19Z)-docosa-4,7,10,13,16,19-hexaenamidoethyl)(2-morpholinoethyl)amino)ethylamino)-4-oxobut-2-enoate (I-29);
(E)-methyl 4-(2-((2-(4Z,7Z,10Z,13Z,16Z,19Z)-docosa-4,7,10,13,16,19-hexaenamidoethyl)(3-(piperazin-1-yl)propyl)amino)ethylamino)-4-oxobut-2-enoate (I-30);
(E)-methyl 4-(3-(4Z,7Z,10Z,13Z,16Z,19Z)-docosa-4,7,10,13,16,19-hexaenamido-2-oxopropylamino)-4-oxobut-2-enoate (I-31);
(E)-methyl 4-(3-(4Z,7Z,10Z,13Z,16Z,19Z)-docosa-4,7,10,13,16,19-hexaenamido-2-morpholinopropylamino)-4-oxobut-2-enoate (I-32);
(E)-methyl 4-(3-(4Z,7Z,10Z,13Z,16Z,19Z)-docosa-4,7,10,13,16,19-hexaenamido-2-(piperazin-1-yl)propylamino)-4-oxobut-2-enoate (I-33);
(E)-methyl 4-(5-(4Z,7Z,10Z,13Z,16Z,19Z)-docosa-4,7,10,13,16,19-hexaenamido-3-hydroxypentylamino)-4-oxobut-2-enoate (I-34);
(E)-methyl 4-(5-(4Z,7Z,10Z,13Z,16Z,19Z)-docosa-4,7,10,13,16,19-hexaenamido-3-morpholinopentylamino)-4-oxobut-2-enoate (I-35);
(E)-methyl 4-(2-(2-(2-(4Z,7Z,10Z,13Z,16Z,19Z)-docosa-4,7,10,13,16,19-hexaenamidoethoxy)ethoxy)ethylamino)-4-oxobut-2-enoate (I-36);
(E)-methyl 4-(2-(2-(4Z,7Z,10Z,13Z,16Z,19Z)-docosa-4,7,10,13,16,19-hexaenamidoethylthio)ethylamino)-4-oxobut-2-enoate (I-37);
(E)-methyl 4-(3-(2-(4Z,7Z,10Z,13Z,16Z,19Z)-docosa-4,7,10,13,16,19-hexaenamidoacetoxy)-1-methoxy-1-oxobutan-2-ylamino)-4-oxobut-2-enoate (I-38);
(E)-methyl 4-((R)-3-(1-(2-(4Z,7Z,10Z,13Z,16Z,19Z)-docosa-4,7,10,13,16,19-hexaenamidoethyl)-2,5-dioxopyrrolidin-3-ylthio)-1-methoxy-1-oxopropan-2-ylamino)-4-oxobut-2-enoate (I-39);
(E)-methyl 4-(4-(4Z,7Z,10Z,13Z,16Z,19Z)-docosa-4,7,10,13,16,19-hexaenoylpiperazin-1-yl)-4-oxobut-2-enoate (I-40);
(E)-methyl 4-((2R,6S)-4-((4Z,7Z,100Z,13Z,16Z,19Z)-docosa-4,7,10,13,16,19-hexaenoyl)-2,6-dimethylpiperazin-1-yl)-4-oxobut-2-enoate (I-41);
(E)-methyl 4-((1S,4S)-5-((4Z,7Z,10Z,13Z,16Z,19Z)-docosa-4,7,10,13,16,19-hexaenoyl)-2,5-diaza-bicyclo[2.2.1]heptan-2-yl)-4-oxobut-2-enoate (I-42);
(E)-methyl 4-((2-((4Z,7Z,10Z,13Z,16Z,19Z)-docosa-4,7,10,13,16,19-hexaenamidomethyl)cyclopropyl)methylamino)-4-oxobut-2-enoate (I-43);
(E)-methyl 4-((4-(4Z,7Z,10Z,13Z,16Z,19Z)-docosa-4,7,10,13,16,19-hexaenamidocyclohexyl)methylamino)-4-oxobut-2-enoate (I-44);
(E)-methyl 4-(4-((4Z,7Z,10Z,13Z,16Z,19Z)-docosa-4,7,10,13,16,19-hexaenamidomethyl)cyclohexylamino)-4-oxobut-2-enoate (I-45);
(E)-methyl 4-(3-(4Z,7Z,10Z,13Z,16Z,19Z)-docosa-4,7,10,13,16,19-hexaenoyl-3-aza-bicyclo[3.1.0]hexan-6-ylamino)-4-oxobut-2-enoate (I-46);
(E)-methyl 4-(6-(4Z,7Z,10Z,13Z,16Z,19Z)-docosa-4,7,10,13,16,19-hexaenamido-3-aza-bicyclo[3.1.0]hexan-3-yl)-4-oxobut-2-enoate (I-47);
(E)-methyl 4-((S)-1-((4Z,7Z,10Z,13Z,16Z,19Z)-docosa-4,7,10,13,16,19-hexaenoyl)pyrrolidin-3-ylamino)-4-oxobut-2-enoate (I-48);
(E)-methyl 4-((S)-3-((4Z,7Z,10Z,13Z,16Z,19Z)-docosa-4,7,10,13,16,19-hexaenamido)pyrrolidin-1-yl)-4-oxobut-2-enoate (I-49);
(E)-methyl 4-((1-(4Z,7Z,10Z,13Z,16Z,19Z)-docosa-4,7,10,13,16,19-hexaenoylpyrrolidin-2-yl)methylamino)-4-oxobut-2-enoate (I-50);
(E)-methyl 4-(2-((4Z,7Z,10Z,13Z,16Z,19Z)-docosa-4,7,10,13,16,19-hexaenamidomethyl)pyrrolidin-1-yl)-4-oxobut-2-enoate (I-51);
(E)-methyl 4-(1-(4Z,7Z,10Z,13Z,16Z,19Z)-docosa-4,7,10,13,16,19-hexaenoylpiperidin-4-ylamino)-4-oxobut-2-enoate (I-52);
(E)-methyl 4-(4-(4Z,7Z,10Z,13Z,16Z,19Z)-docosa-4,7,10,13,16,19-hexaenamidopiperidin-1-yl)-4-oxobut-2-enoate (I-53);
(E)-methyl 4-((1-(4Z,7Z,10Z,13Z,16Z,19Z)-docosa-4,7,10,13,16,19-hexaenoylpiperidin-4-yl)methylamino)-4-oxobut-2-enoate (I-54);
(E)-methyl 4-(4-((4Z,7Z,10Z,13Z,16Z,19Z)-docosa-4,7,10,13,16,19-hexaenamidomethyl)piperidin-1-yl)-4-oxobut-2-enoate (I-55);
(E)-methyl 4-((1-(4Z,7Z,10Z,13Z,16Z,19Z)-docosa-4,7,10,13,16,19-hexaenoylpiperidin-2-yl)methylamino)-4-oxobut-2-enoate (I-56);
(E)-methyl 4-(2-((4Z,7Z,10Z,13Z,16Z,19Z)-docosa-4,7,10,13,16,19-hexaenamidomethyl)piperidin-1-yl)-4-oxobut-2-enoate (I-57);
(E)-methyl 4-((4-(4Z,7Z,10Z,13Z,16Z,19Z)-docosa-4,7,10,13,16,19-hexaenoylmorpholin-3-yl)methylamino)-4-oxobut-2-enoate (I-58);
(E)-methyl 4-(3-((4Z,7Z,10Z,13Z,16Z,19Z)-docosa-4,7,10,13,16,19-hexaenamidomethyl)morpholino)-4-oxobut-2-enoate (I-59);
(E)-methyl 4-(5-(4Z,7Z,10Z,13Z,16Z,19Z)-docosa-4,7,10,13,16,19-hexaenoyl-hexahydropyrrolo[3,4-c]pyrrol-2(1H)-yl)-4-oxobut-2-enoate (I-60);
(E)-methyl 4-(1-(4Z,7Z,10Z,13Z,16Z,19Z)-docosa-4,7,10,13,16,19-hexaenoyl-hexahydropyrrolo[3,4-b]pyrrol-5(1H)-yl)-4-oxobut-2-enoate (I-61);
(E)-methyl 4-((2-(4Z,7Z,10Z,13Z,16Z,19Z)-docosa-4,7,10,13,16,19-hexaenoyloctahydropyrrolo[1,2-a]pyrazin-7-yl)methylamino)-4-oxobut-2-enoate (I-62);
(E)-methyl 4-(7-((4Z,7Z,10Z,13Z,16Z,19Z)-docosa-4,7,10,13,16,19-hexaenamidomethyl)-hexahydropyrrolo[1,2-a]pyrazin-2(1H)-yl)-4-oxobut-2-enoate (I-63);
(E)-methyl 4-(4-((4Z,7Z,10Z,13Z,16Z,19Z)-docosa-4,7,10,13,16,19-hexaenamidomethyl)phenylamino)-4-oxobut-2-enoate (I-64);
(E)-methyl 4-(6-((4Z,7Z,10Z,13Z,16Z,19Z)-docosa-4,7,10,13,16,19-hexaenamidomethyl)pyridin-2-ylamino)-4-oxobut-2-enoate (I-65);
(E)-ethyl 4-(2-(4Z,7Z,10Z,13Z,16Z,19Z)-docosa-4,7,10,13,16,19-hexaenamidoethylamino)-4-oxobut-2-enoate (I-66);
(E)-ethyl 4-(2-((2-(4Z,7Z,10Z,13Z,16Z,19Z)-docosa-4,7,10,13,16,19-hexaenamidoethyl)(methyl)amino)ethylamino)-4-oxobut-2-enoate (I-67);
(E)-ethyl 4-(2-(2-(4Z,7Z,10Z,13Z,16Z,19Z)-docosa-4,7,10,13,16,19-hexaenamidoethylamino)ethylamino)-4-oxobut-2-enoate (I-68);
(S)-6-((4Z,7Z,10Z,13Z,16Z,19Z)-docosa-4,7,10,13,16,19-hexaenamido)-2-((E)-4-ethoxy-4-oxobut-2-enamido)hexanoic acid (I-69);
(S)-2-((4Z,7Z,100Z,13Z,16Z,19Z)-docosa-4,7,10,13,16,19-hexaenamido)-6-((E)-4-ethoxy-4-oxobut-2-enamido)hexanoic acid (I-70);
(S)-6-((5Z,8Z,11Z,14Z,17Z)-icosa-5,8,11,14,17-pentaenamido)-2-((E)-4-methoxy-4-oxobut-2-enamido)hexanoic acid (I-71);
(S)-2-((5Z,8Z,11Z,14Z,17Z)-icosa-5,8,11,14,17-pentaenamido)-6-((E)-4-methoxy-4-oxobut-2-enamido)hexanoic acid (I-72);
(E)-methyl 4-(2-(2-(5Z,8Z,11Z,14Z,17Z)-icosa-5,8,11,14,17-pentaenamidoethylamino)ethylamino)-4-oxobut-2-enoate (I-73);
(E)-methyl 4-(2-((2-(5Z,8Z,11Z,14Z,17Z)-icosa-5,8,11,14,17-pentaenamidoethyl)(methyl)amino)ethylamino)-4-oxobut-2-enoate (I-74);
(E)-ethyl 4-(2-(5Z,8Z,11Z,14Z,17Z)-icosa-5,8,11,14,17-pentaenamidoethylamino)-4-oxobut-2-enoate (I-75);
(S)-2-((E)-4-ethoxy-4-oxobut-2-enamido)-6-((5Z,8Z,11Z,14Z,17Z)-icosa-5,8,11,14,17-pentaenamido)hexanoic acid (I-76);
(S)-6-((E)-4-ethoxy-4-oxobut-2-enamido)-2-((5Z,8Z,11Z,14Z,17Z)-icosa-5,8,11,14,17-pentaenamido)hexanoic acid (I-77);
(E)-ethyl 4-(2-((2-(5Z,8Z,11Z,14Z,17Z)-icosa-5,8,11,14,17-pentaenamidoethyl)(methyl)amino)ethylamino)-4-oxobut-2-enoate (I-78);
(E)-ethyl 4-(2-(2-(5Z,8Z,11Z,14Z,17Z)-icosa-5,8,11,14,17-pentaenamidoethylamino)ethylamino)-4-oxobut-2-enoate (I-79);
(S)-5-((4Z,7Z,10Z,13Z,16Z,19Z)-docosa-4,7,10,13,16,19-hexaenamido)-2-((E)-4-methoxy-4-oxobut-2-enamido)pentanoic acid (I-80);
(S)-2-((4Z,7Z,10Z,13Z,16Z,19Z)-docosa-4,7,10,13,16,19-hexaenamido)-5-((E)-4-methoxy-4-oxobut-2-enamido)pentanoic acid (I-81);
(S)-1,3-dihydroxypropan-2-yl 5-((4Z,7Z,10Z,13Z,16Z,19Z)-docosa-4,7,10,13,16,19-hexaenamido)-2-((E)-4-methoxy-4-oxobut-2-enamido)pentanoate (I-82);
(S)-1,3-dihydroxypropan-2-yl 2-((4Z,7Z,10Z,13Z,16Z,19Z)-docosa-4,7,10,13,16,19-hexaenamido)-5-((E)-4-methoxy-4-oxobut-2-enamido)pentanoate (I-83);
(S)-5-((5Z,8Z,11Z,14Z,17Z)-icosa-5,8,11,14,17-pentaenamido)-2-((E)-4-methoxy-4-oxobut-2-enamido)pentanoic acid (I-84);
(S)-2-((5Z,8Z,11Z,14Z,17Z)-icosa-5,8,11,14,17-pentaenamido)-5-((E)-4-methoxy-4-oxobut-2-enamido)pentanoic acid (I-85);
(S)-1,3-dihydroxypropan-2-yl 5-((5Z,8Z,11Z,14Z,17Z)-icosa-5,8,11,14,17-pentaenamido)-2-((E)-4-methoxy-4-oxobut-2-enamido)pentanoate (I-86);
(S)-1,3-dihydroxypropan-2-yl 2-((5Z,8Z,11Z,14Z,17Z)-icosa-5,8,11,14,17-pentaenamido)-5-((E)-4-methoxy-4-oxobut-2-enamido)pentanoate (I-87);
(S)-5-((4Z,7Z,10Z,13Z,16Z,19Z)-docosa-4,7,10,13,16,19-hexaenamido)-2-((E)-4-ethoxy-4-oxobut-2-enamido)pentanoic acid (I-88);
(S)-2-((4Z,7Z,10Z,13Z,16Z,19Z)-docosa-4,7,10,13,16,19-hexaenamido)-5-((E)-4-ethoxy-4-oxobut-2-enamido)pentanoic acid (I-89);
(S)-1,3-dihydroxypropan-2-yl 5-((4Z,7Z,10Z,13Z,16Z,19Z)-docosa-4,7,10,13,16,19-hexaenamido)-2-((E)-4-ethoxy-4-oxobut-2-enamido)pentanoate (I-90);
(S)-1,3-dihydroxypropan-2-yl 2-((4Z,7Z,10Z,13Z,16Z,19Z)-docosa-4,7,10,13,16,19-hexaenamido)-5-((E)-4-ethoxy-4-oxobut-2-enamido)pentanoate (I-91);
(S)-2-((E)-4-ethoxy-4-oxobut-2-enamido)-5-((5Z,8Z,11Z,14Z,17Z)-icosa-5,8,11,14,17-pentaenamido)pentanoic acid (I-92);
(S)-5-((E)-4-ethoxy-4-oxobut-2-enamido)-2-((5Z,8Z,11Z,14Z,17Z)-icosa-5,8,11,14,17-pentaenamido)pentanoic acid (I-93);
(S)-1,3-dihydroxypropan-2-yl 2-((E)-4-ethoxy-4-oxobut-2-enamido)-5-((5Z,8Z,11Z,14Z,17Z)-icosa-5,8,11,14,17-pentaenamido)pentanoate (I-94);
(S)-1,3-dihydroxypropan-2-yl 5-((E)-4-ethoxy-4-oxobut-2-enamido)-2-((5Z,8Z,11Z,14Z,17Z)-icosa-5,8,11,14,17-pentaenamido)pentanoate (I-95);
(S)-1,3-dihydroxypropan-2-yl 6-((5Z,8Z,11Z,14Z,17Z)-icosa-5,8,11,14,17-pentaenamido)-2-((E)-4-methoxy-4-oxobut-2-enamido)hexanoate (I-96);
(S)-1,3-dihydroxypropan-2-yl 2-((5Z,8Z,11Z,14Z,17Z)-icosa-5,8,11,14,17-pentaenamido)-6-((E)-4-methoxy-4-oxobut-2-enamido)hexanoate (I-97);
(S)-1,3-dihydroxypropan-2-yl 6-((4Z,7Z,10Z,13Z,16Z,19Z)-docosa-4,7,10,13,16,19-hexaenamido)-2-((E)-4-ethoxy-4-oxobut-2-enamido)hexanoate (I-98);
(S)-1,3-dihydroxypropan-2-yl 2-((E)-4-ethoxy-4-oxobut-2-enamido)-6-((5Z,8Z,11Z,14Z,17Z)-icosa-5,8,11,14,17-pentaenamido)hexanoate (I-99);
(S)-1,3-dihydroxypropan-2-yl 2-((4Z,7Z,10Z,13Z,16Z,19Z)-docosa-4,7,10,13,16,19-hexaenamido)-6-((E)-4-ethoxy-4-oxobut-2-enamido)hexanoate (I-100);
(S)-1,3-dihydroxypropan-2-yl 6-((E)-4-ethoxy-4-oxobut-2-enamido)-2-((5Z,8Z,11Z,14Z,17Z)-icosa-5,8,11,14,17-pentaenamido)hexanoate (I-101);
(E)-4-(2-(4Z,7Z,10Z,13Z,16Z,19Z)-docosa-4,7,10,13,16,19-hexaenamidoethylamino)-4-oxobut-2-enoic acid (I-102);
2-(4Z,7Z,10Z,13Z,16Z,19Z)-docosa-4,7,10,13,16,19-hexaenamidoethyl methyl fumarate (I-103);
(E)-methyl 4-(methyl(2-((4Z,7Z,10Z,13Z,16Z,19Z)—N-methyldocosa-4,7,10,13,16,19-hexaenamido)ethyl)amino)-4-oxobut-2-enoate (I-104);
(R,E)-methyl 4-(2-(5-(1,2-dithiolan-3-yl)pentanamido)ethylamino)-4-oxobut-2-enoate (I-105);
6-(5-((R)-1,2-dithiolan-3-yl)pentanamido)-2-((E)-4-methoxy-4-oxobut-2-enamido)hexanoic acid (I-106);
2-(5-((R)-1,2-dithiolan-3-yl)pentanamido)-6-((E)-4-methoxy-4-oxobut-2-enamido)hexanoic acid (I-107);
(R,E)-methyl 4-(2-(2-(5-(1,2-dithiolan-3-yl)pentanamido)ethylamino)ethylamino)-4-oxobut-2-enoate (I-108);
(R,E)-methyl 4-(2-((2-(5-(1,2-dithiolan-3-yl)pentanamido)ethyl)(methyl)amino)ethylamino)-4-oxobut-2-enoate (I-109);
N¹,N⁴-bis(2-(4Z,7Z,10Z,13Z,16Z,19Z)-docosa-4,7,10,13,16,19-hexaenamidoethyl)fumaramide (II-1); and
N¹-(2-(4Z,7Z,10Z,13Z,16Z,19Z)-docosa-4,7,10,13,16,19-hexaenamidoethyl)-N4-(2-(5Z,8Z,11Z,14Z,17Z)-icosa-5,8,11,14,17-pentaenamidoethyl)fumaramide (11-2).

### Methods of Making Fatty Acid Fumarate Derivatives

FAFDs of Formula Formula I, IA, IB, IC and II disclosed herein may be obtained via the synthetic methods described in U.S. Pat. No. 8,969,354, the entire disclosure of which is incorporated herein by reference. General synthetic methods useful in the synthesis of compounds described herein are available in the art.

### Pharmaceutical Compositions

The invention also includes pharmaceutical compositions useful for treating or preventing a heart failure disease, including HFPEF. The compositions may be suitable for internal use and comprise an effective amount of a FAFD and a pharmaceutically acceptable carrier. The FAFDs are especially useful in that they demonstrate very low peripheral toxicity or no peripheral toxicity.

Administration of the FAFDs can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in a solid, semi-solid, or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a FAFD and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the FAFD is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the FAFDs.

The FAFDs can be also formulated as a suppository that can be prepared from fatty emulsions or suspensions, doe example using polyalkylene glycols such as propylene glycol, as the carrier.

The FAFDs can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the entire disclosure of which is herein incorporated by reference.

FAFDs can also be delivered by the use of monoclonal antibodies as individual carriers to which the FAFDs are coupled. The FAFDs can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the FAFDs can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, FAFDs are not covalently bound to a polymer, e.g., a polycarboxylic acid polymer, or a polyacrylate.

Parenteral injectable administration is generally used for subcutaneous, intramuscular, or intravenous injections and infusions. Injectables can be prepared in conventional forms, either as liquid solutions or suspensions or solid forms suitable for dissolving in liquid prior to injection.

Compositions can be prepared according to conventional mixing, granulating, or coating methods, respectively, and the present pharmaceutical compositions can contain from about 0.1% to about 80%, from about 5% to about 60%, or from about 1% to about 20% of the FAFD by weight or volume.

### Methods of Using Fatty Acid Fumarate Derivatives

Also provided in the present disclosure is a method for inhibiting, preventing, or treating a heart failure disease in a subject by administration of a FAFD of Formula I, IA, IB, IC, and II.

For example, the heart failure disease is heart failure with preserved ejection fraction (HFPEF).

For example, the heart failure disease is heart failure with an ejection fraction ≥40%.

For example, the heart failure disease is diastolic heart failure.

For example, the heart failure disease is heart failure with increased levels of TNF-α, IL-6, CRP, or other pro-inflammatory cytokines

For example, the heart failure disease is hypertension with risk of developing HFPEF.

For example, the heart failure disease is atrial fibrillation with risk of developing HFPEF.

For example, the heart failure disease is diabetes with risk of developing HFPEF.

For example, the heart failure disease is COPD with risk of developing HFPEF.

For example, the heart failure disease is ischemic heart disease with risk of developing HFPEF.

For example, the heart failure disease is obesity with risk of developing HFPEF.

For example, the heart failure disease is chronic heart failure.

For example, the heart failure disease is compensated heart failure.

For example, the heart failure disease is decompensated heart failure.

For example, the heart failure disease is a condition that has a high risk of developing HFPEF.

In one embodiment, the present disclosure provides a method of treating a heart failure disease in a subject in need thereof, the method comprising administering to the subject a therapeutically effective amount of one or more FAFDs of Formula I

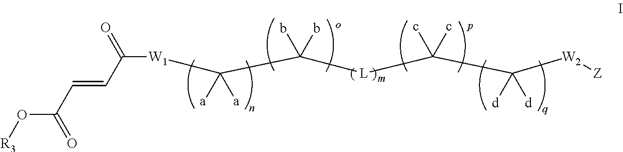

or a pharmaceutically acceptable salt, hydrate, enantiomer, or stereoisomer thereof;

wherein

each W1 and W2 is independently null, O, S, NH, or NR, or W1 and W2 can be taken together to form an optionally substituted imidazolidine or piperazine group;

each a, b, c, and d, is independently —H, -D, —CH3, —OCH3, —OCH2CH3, —C(O)OR, —O—Z, or benzyl, or two of a, b, c, and d can be taken together, along with the single carbon to which they are bound, to form a cycloalkyl or heterocycle;

each n, o, p, and q is independently 0, 1, or 2;

each L is independently null, —O—, —C(O)—, —S—, —S(O)—, —S(O)2-, —S—S—, —(C1-C6 alkyl)-, —(C3-C6 cycloalkyl)-, a heterocycle, a heteroaryl,

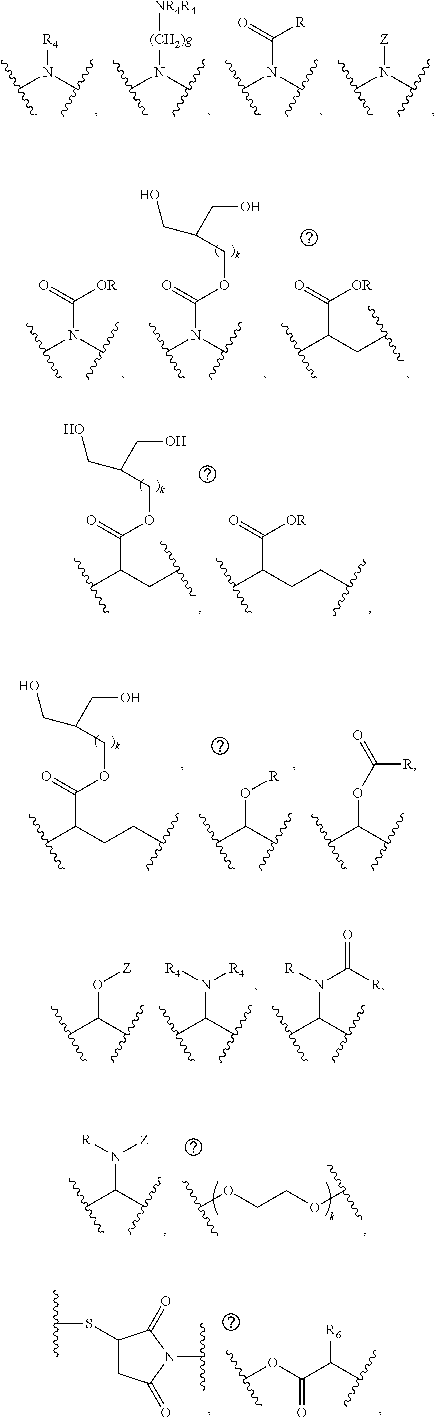

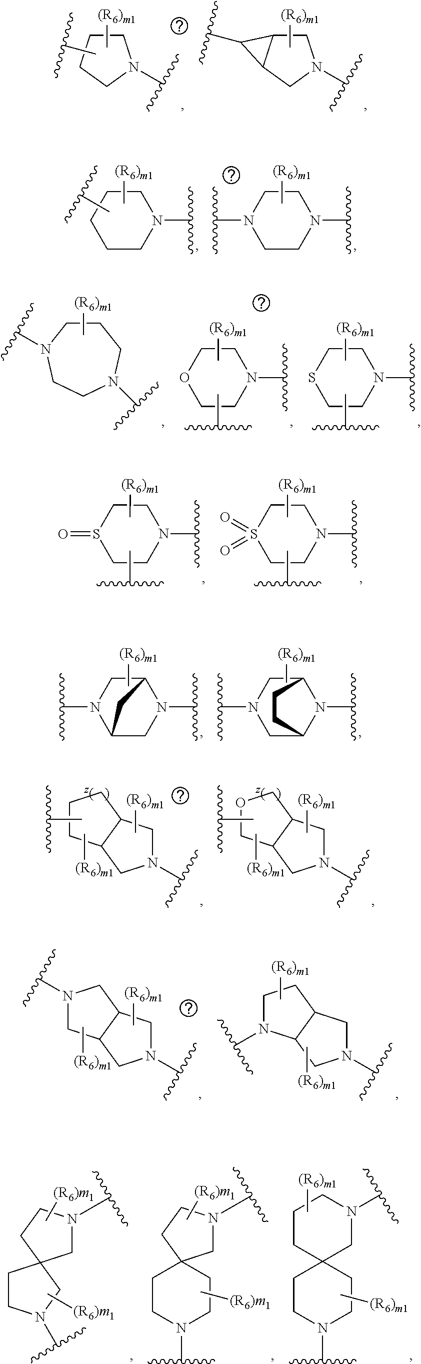

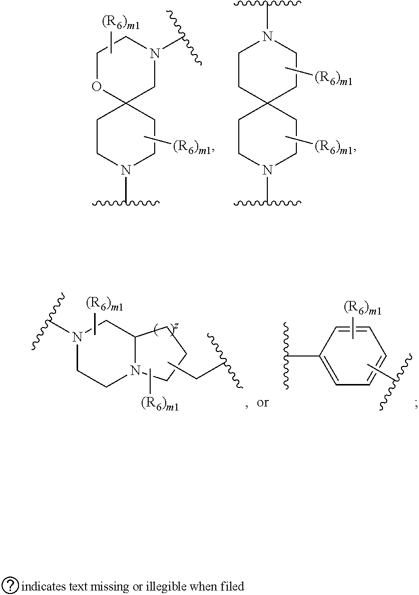

wherein the representation of L is not limited directionally left to right as is depicted, rather either the left side or the right side of L can be bound to the W1 side of the compound of Formula I;

each R₆ is independently —H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₂-C₃ alkene, —C₂-C₃ alkyne, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C1-C₃ alkyl), —S(O)C1-C₃ alkyl, or —S(O)₂C1-C₃ alkyl;

each g is independently 2, 3, or 4;

each h is independently 1, 2, 3, or 4;

each m is independently 0, 1, 2, or 3; if m is more than 1, then L can be the same or different;

each m1 is independently 0, 1, 2, or 3;

k is 0, 1, 2, or 3;

z is 1, 2, or 3;

each R₄ is independently H or optionally substituted C₁-C₆ alkyl, wherein a methylene unit of the C₁-C₆ alkyl can be optionally substituted for either O or NR, and in NR₄R4, both R₄ when taken together with the nitrogen to which they are attached can form a heterocyclic ring such as a pyrrolidine, piperidine, morpholine, piperazine or pyrrole;

each Z is independently H,

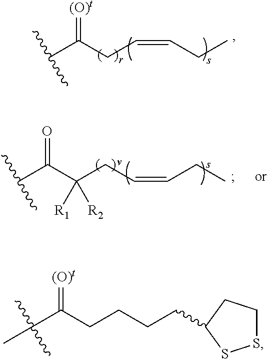

provided that there is at least one

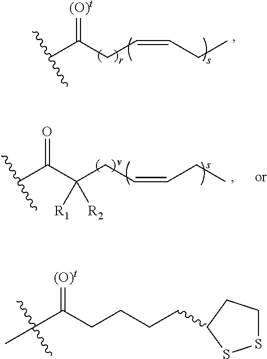

in the compound;

each t is independently 0 or 1;

each r is independently 2, 3, or 7;

each s is independently 3, 5, or 6;

each v is independently 1, 2, or 6;

each R₁ and R₂ is independently —H, -D, —C₁-C₄ alkyl, -halogen, —OH, —C(O)C1-C₄ alkyl, —O-aryl, —O-benzyl, —OC(O)C₁-C₄ alkyl, —C₂-C₃ alkene, —C₂-C₃ alkyne, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

each R₃ is independently H, or —C₁-C₆ alkyl;

each R₅ is independently e, H, or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each R is independently —H, —C₁-C₃ alkyl, or straight or branched C₁-C₄ alkyl optionally substituted with OH, or halogen;

provided that when each of m, n, o, p, and q is 0, W₁ and W₂ are each null, and Z is

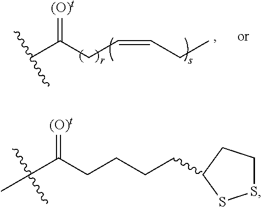

then t must be 0; and

when each of m, n, o, p, and q is 0, and W1 and W₂ are each null, then Z must not be

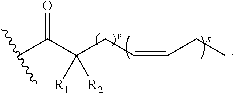

In one embodiment, the present disclosure provides method of treating a heart failure disease in a subject in need thereof, the method comprising administering to the subject a therapeutically effective amount of FAFD of formula

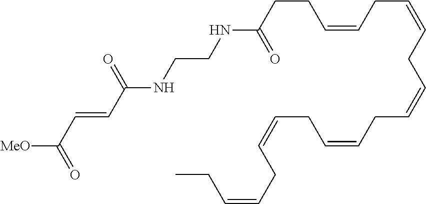 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof.

In certain embodiments, the heart failure disease may be heart failure with preserved ejection fraction (HFPEF); heart failure with ejection fraction ≥40%; diastolic heart failure; heart failure with elevated levels of TNF-α, IL-6, CRP, or TGF-β; hypertension with a risk of developing HFPEF; atrial fibrillation with a risk of developing HFPEF; diabetes with a risk of developing HFPEF; COPD with a risk of developing HFPEF; ischemic heart disease with a risk of developing HFPEF; obesity with a risk of developing HFPEF; chronic heart failure; compensated heart failure; decompensated heart failure; or other conditions known to have a high risk of developing HFPEF. In particular, heart failure disease is heart failure with preserved ejection fraction (HFPEF).

FAFDs may be assayed in vitro and in vivo for the desired therapeutic or prophylactic activity prior to use in humans. In vivo assays, for example using appropriate animal models, may also be used to determine whether administration of a FAFD is therapeutically effective.

### Dosage and Administration

In some embodiments, the subject is administered an effective amount of a FAFD.

The dosage regimen utilizing the FAFD is selected in accordance with a variety of factors including type, species, age, weight, sex and medical condition of the subject; the severity of the condition to be treated; the route of administration; the renal or hepatic function of the subject; and the particular FAFD employed. A physician or veterinarian of ordinary skill in the art can readily determine and prescribe the effective amount of the drug required to prevent, counter, or arrest the progress of the condition.

In addition, in vitro or in vivo assays may be employed to help identify optimal dosage ranges. For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

Effective dosage amounts of the present invention, when used for the indicated effects, range from about 20 mg to about 5000 mg of the FAFD per day. Compositions for in vivo or in vitro use can contain about 20, 50, 75, 100, 150, 250, 500, 750, 1000, 1250, 2500, 3500, or 5000 mg of the FAFD. In one embodiment, the composition is in the form of a tablet that can be scored. Effective plasma levels of the FAFD can range from about 0.002 mg to about 100 mg per kg of body weight per day. Appropriate dosages of the FAFDs can be determined as set forth in Goodman, L. S.; Gilman, A. The Pharmacological Basis of Therapeutics, 5th ed.; MacMillan: New York, 1975, pp. 201-226.

FAFDs can be administered in a single daily dose, or the total daily dosage can be administered in divided doses of two, three or four times daily. Furthermore, FAFDs can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays, and gels, wherein the concentration of the FAFD ranges from about 0.1% to about 15%, w/w or w/v.

In certain embodiments, a therapeutically effective dose of a FAFD may provide therapeutic benefit without causing substantial toxicity including adverse side effects. Toxicity of FAFD and/or metabolites thereof may be determined using standard pharmaceutical procedures and may be ascertained by those skilled in the art. The dose ratio between toxic and therapeutic effect is the therapeutic index. A dose of a FAFD may be within a range capable of establishing and maintaining a therapeutically effective circulating plasma and/or blood concentration of a FAFD that exhibits little or no toxicity. A dose may vary within this range depending upon the dosage form employed and the route of administration utilized. In certain embodiments, an escalating dose may be administered.

The dose will be adjusted to the individual requirements in each particular case. That dosage may vary within wide limits depending upon numerous factors such as the severity of the disease to be treated, the age and general health condition of the subject, other medicaments with which the subject is being treated, the route and form of administration, and the preferences and experience of the medical practitioner involved.

For oral administration, therapeutically effective amount of

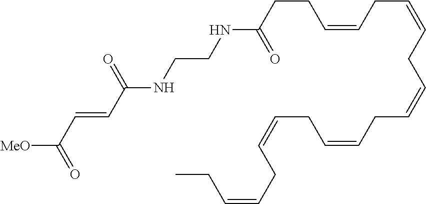 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof that is shown to provide MMF plasma exposure comparable to dimethyl fumarate (DMF) 120 mg to 720 mg per day as a monotherapy and/or in combination therapy.

In one embodiment, daily dose comprises about 20 mg to about 5000 mg of

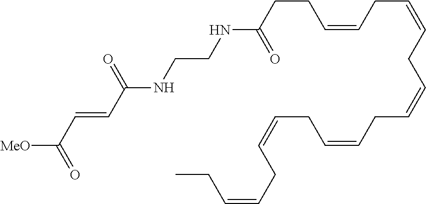 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof.

One of ordinary skill in treating diseases described herein will be able, without undue experimentation and in reliance on personal knowledge, experience and the disclosures of this application, to ascertain a therapeutically effective amount of the compounds of the present invention for a given disease and subject.

### Combination Therapy

Methods provided by the present disclosure further comprise administering one or more pharmaceutically active compounds in addition to one or more a FAFDs.

Such compounds may be provided to treat the same disease or a different disease than the disease being treated with the FAFDs.

In certain embodiments, FAFDs may be used in combination with at least one other therapeutic agent. In certain embodiments, FAFDs may be administered to a subject together with another compound for treating a heart failure disease, such as HFPEF.

FAFDs and the at least one other therapeutic agent may act additively or, and in certain embodiments, synergistically. The at least one additional therapeutic agent may be included in the same dosage form as FAFDs or may be provided in a separate dosage form. Methods provided by the present disclosure can further include, in addition to administering a FAFD, administering one or more therapeutic agents effective for treating the same or different disease than the disease being treated by a FAFDs. Methods provided by the present disclosure include administration of a FAFD and one or more other therapeutic agents provided that the combined administration does not inhibit the therapeutic efficacy of the FAFD and/or does not typically produce significant and/or substantial adverse combination effects.

In certain embodiments, dosage forms comprising FAFDs may be administered concurrently with the administration of another therapeutic agent, which may be part of the same dosage form as, or in a different dosage form than that comprising a FAFD.

A FAFD may be administered prior or subsequent to administration of another therapeutic agent. In certain embodiments of combination therapy, the combination therapy may comprise alternating between administering a FAFD and a composition comprising another therapeutic agent, e.g., to minimize adverse drug effects associated with a particular drug. When a FAFD is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising a FAFD may be administered with one or more substances to enhance, modulate and/or control release, bioavailability, therapeutic efficacy, therapeutic potency, stability, and the like of a FAFD. For example, to enhance the therapeutic efficacy of a FAFD, the FAFD may be co-administered with or a dosage form comprising a FAFD may comprise one or more active agents to increase the absorption or diffusion of a FAFD from the gastrointestinal tract to the systemic circulation, or to inhibit degradation of the FAFD in the blood of a subject. In certain embodiments, a FAFD may be co-administered with an active agent having pharmacological effects that enhance the therapeutic efficacy of a FAFD.

In certain embodiments, FAFDs provided by the present disclosure and pharmaceutical compositions thereof may be administered to a subject for treating heart failure with preserved ejection fraction (HFPEF) in combination with a therapy or another therapeutic agent known or believed to be effective in treating HFPEF.

In certain embodiments, administration of FAFD may also be carried out in the combination with administration of one or more preparations of a second agent useful for treating heart failure, such as but not limited to diuretics, ace-inhibitors, beta-blockers, angiotensin receptor blockers, isosorbide dinitrate, hydralazine, angiotensin receptor-neprilysin inhibitors, aldosterone antagonists, a PDE5 inhibitor, a statin, a neprilysin inhibitor, an aldosterone inhibitor, or an antitumor necrosis factor-alpha therapy. In one embodiment, the second agent is a statin, for example atorvastatin, fluvastatin, lovastatin, pravastatin, rosuvastatin, or simvastatin. For this purpose, the preparations administered may comprise a combination of the active ingredients in the known dosages or amounts, respectively.

In certain embodiments, FAFDs provided by the present disclosure and pharmaceutical compositions thereof may be administered to a subject for treating heart failure with preserved ejection fraction (HFPEF) in combination with a statin (HMG-CoA reductase inhibitor).

In one embodiment, combination relates to (a)

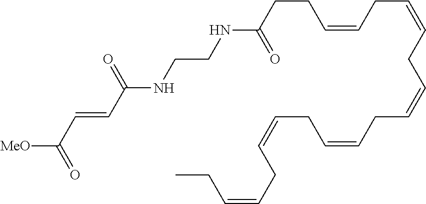 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof and (b) a statin.

In some embodiments, a pharmaceutical composition is provided comprising

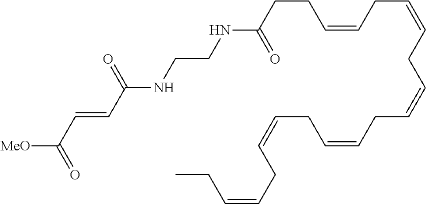 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof and (b) a statin and one or more pharmaceutically acceptable excipients.

In one embodiment,

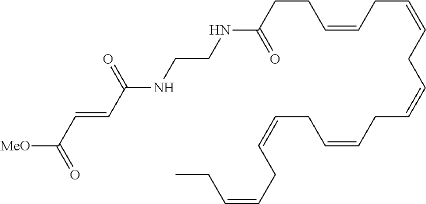 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof at a dose range of about 20 mg to about 5000 mg of the FAFD per day and the statin at a dose range of 10 mg to 80 mg.

In certain embodiments, FAFDs provided by the present disclosure and pharmaceutical compositions thereof may be administered to a subject for treating heart failure with preserved ejection fraction (HFPEF) in combination with an aldosterone antagonist.

In certain embodiments, FAFDs provided by the present disclosure and pharmaceutical compositions thereof may be administered to a subject for treating heart failure with preserved ejection fraction (HFPEF) in combination with an angiotensin-receptor neprilysin inhibitor (ARNI).

In certain embodiments, FAFDs provided by the present disclosure and pharmaceutical compositions thereof may be administered to a subject for treating heart failure with reduced ejection fraction (HFREF) in combination with a therapy or another therapeutic agent known or believed to be effective in treating HFREF. Useful drugs for treating HFREF include antitensin-modulating agents, diuretics such as furosemide, bumetanie, hydrochlorothiazide, chlorthalidone, chlorthiazide, spironolactone, eplerenone: beta blockers such as bisoprolol, carvedilol, and metroprolol; positive inotropes such as digoxin, milrinone, and dobutamine; alternative vasodilators such as isosorbide dinitrate/hydralazine; aldosterone receptor antagonists; recombinant neuroendocrine hormones such as nesiritide; angiotensin receptor-neprilysin inhibitors such as LCZ696; and vasopressin receptor antagonists such as tolvaptan and conivaptan.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### Prophetic Example 4

The following prophetic example serves to provide approximate dosage levels of FAFDs to achieve the intended effect, for example treatment of heart failure with preserved ejection fraction (HFPEF). Based on the literature, a few assumptions about the dosage can be made, as will be described in further detail below.

Docosahexaenoic acid (DHA) or pharmaceutically acceptable salts, esters, amides, epoxides, and prodrugs thereof have been used by Timothy O'Connell et. al. for treating or limiting development of heart failure with preserved ejection fraction (HFPEF). The pharmaceutical composition comprises purified or synthesized DHA or a pharmaceutically acceptable salt, ester, amide, epoxide, or prodrug thereof. In certain other embodiments, DHA, or a pharmaceutically acceptable salt, ester, amide, epoxide, or prodrug thereof, is administered to the subject at a concentration from about 5 mg/kg of body weight/day to about 50 mg/kg of body weight/day. In certain other embodiments, the dose is about 600 mg/day to about 1000 mg/day of DHA, or in certain other embodiments, about 800 mg/day.

The full mechanism of fumaric acid esters such as dimethyl fumarate (DMF) and its primary metabolite, monomethyl fumarate (MMF), is not completely understood, but their beneficial effects appear to be mediated, at least in part, through the activation of the NRF2 antioxidant response pathway, which further increases expression of ARE, which increases expression of detoxifying enzymes and antioxidant proteins. For example, a recent publication showed, using ribonucleic acid (RNA) and protein, that CAT-4001, a MMF, inihibits NFkB and activates Nrf2 in vitro and in patient cells (Milne J, et al., “Catabasis Investor Day,” Nov. 17, 2016, New York City, pp. 39-40, the disclosure of which is incorporated by reference in its entirety).

Further, NRF2 deficiency, demonstrated by NRF2 knockout in murine models, results in an earlier onset of cardiac dysfunction induced by pressure and volume overload (Li et al Arterioscler Thromb Vasc Biol. 2009, 29(11), 1843-50). Certain NRF2 activators such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α-estradiol have been used as therapeutic targets to reduce cardiac remodeling, but prodrugs of monomethyl fumarate/FAFDs have not been used yet to reduce cardiac remodeling (Zhou et al; J Appl Physiol. 2015, 119(8), 944-951).

Fumarates are cardioprotective in acute situations via activation of the NRF2 pathway in acute ischemia due to myocardial infarction (Ashrafian et. al; Cell Metab. 2012, 15(3), 361-71). However, Ashrafian et. al claims that fumarates are harmful in chronic situations, including heart failure. FAFDs (molecular conjugate of fumaric acid and fatty acid) are herein proposed to achieve the intended effect, for example, treatment of chronic heart failure with preserved ejection fraction (HFPEF).

Dimethyl Fumarate has been tested for multiple sclerosis and psoriasis at multiple dosages in the past, including 120 mg, 240 mg, daily, BID, and TID. The side effect profile was similar regardless of which dosage was used.

Further, in order to determine dosage of a FAFD, a dose escalation study may be conducted to find a comparable dosage of the FAFD to DMF's 240 mg dose, by comparing plasma levels of MMF. For example, one FAFDs known as (I-1), a compound having formula:

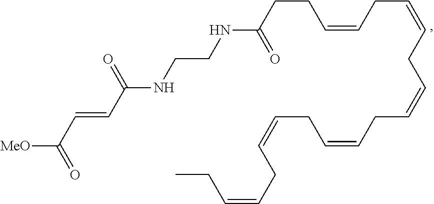 [SMILES: CC/C=C\C/C=C\C/C=C\C/C=C\C/C=C\C/C=C\CCC(=O)NCCNC(=O)/C=C/C(=O)OC]

will be tested to find a comparable dose of 240 mg DMF (Tecfidera) by comparing plasma levels of MMF. Various dosages of a FAFD will be tested in HFPEF subjects so that the dosage that is comparable to a DMF dosage of 120 mg, 240 mg, daily, BID, and TID may be determined more precisely. Using (I-1) as one such FAFD, such dosage is likely to be calculated as near about 20 mg to about 5000 mg of the FAFD per day. In some embodiments, such dosage may contain about 20, 50, 75, 100, 150, 250, 500, 750, 1000, 1250, 2500, 3500, or 5000 mg of the FAFD. In one embodiment, the composition is in the form of a tablet that can be scored. Effective plasma levels of the FAFD can range from about 0.002 mg to about 100 mg per kg of body weight per day.

Furthermore, pro-inflammatory cytokines IL-6 and TNF-α are raised in HFPEF, which may lead to increase activity of VCAM, E-Selection, and NADPH oxidase, which increase ROS in coronary microvasculature endothelial cells, leading to the hallmarks of HFPEF: ventricular stiffness, impaired relaxation, and cardiac dysfunction. The FAFD may reduce damage of ROS in heart failure by multiple pathways including increasing the NRF2/ARE pathway, and possibly by reducing NF-kB, which reduces IL-6 and TNF-α. In some embodiments, treating heart failure includes increasing NFR2/ARE pathway activity.

LCZ696, a combined angiotensin receptor neprilysin inhibitor (ARNI) that has recently shown to reduce mortality in HFREF but not in HFPEF subjects. LCZ696 inhibits natriuretic peptide breakdown and enhances cGMP activation, and in HFPEF was associated with incremental reductions in circulating N-terminal pro-B-type natriuretic peptide (NT-proBNP) levels when compared to treatment with the ARB valsartan, alone. However, these reductions were incremental, and it is yet to be seen whether LCZ696 or other angiotensin receptor-neprilysin inhibitors will lead to any significant mortality or clinical benefit in HFPEF subjects. Furthermore, the comparison with ARB valsartan alone, is flawed in that ARB valsartan is used in the treatment of HFREF but not in HFPEF.

The subjects' baseline TNF-alpha, IL-6, NT-proBNP will be measured at the start of the trial and compared to levels at various intervals (weeks to months to years) to determine the ideal dosage based on reductions in TNF-alpha, IL-6, and/or NT-proBNP. Such a dosage will then be tested in a larger group of HFPEF subjects to measure changes in morbidity and mortality. Thus, an ideal dosage of a FAFD for treating HFPEF will be comparable to a dosage of 120 mg or 240 mg, daily, BID, or TID of DMF (Tecfidera), by measuring MMF concentrations in the blood. In the case of compound (I-1), this dosage range may likely be near about 20 mg to about 5000 mg of the FAFD per day, however, the exact dosage will be determined in subjects based on the description above.

### Prophetic Example 5

Based on the above prophetic example, an exemplary, non-limiting embodiment is described in detail below. As described herein, a user may include a male or female between the ages of 50 to 100 with ejection fraction of greater than 40%, and more likely to be a female with a documented history of high blood pressure, obesity, diabetes, renal disease and/or COPD, with at least one episode of fluid overload, or who has HFPEF or is at risk of developing (HFPEF).

The most common disease leading to HFPEF is systolic hypertension, which is present in more than 85% of subjects. Subjects with HFPEF have normal left ventricular (LV) end-diastolic volume and normal (or near-normal) EF and stroke volume and commonly exhibit concentric remodeling of either the LV chamber and/or cardiomyocytes.

Subjects with HFPEF have a devastating 5-year mortality rate (approaching 60%), costly morbidity (6-month hospitalization rate of 50%), and debilitating symptoms (maximum myocardial oxygen consumption [MVo₂] averaging 14 mL/g/min).

More than half of heart failure subjects have heart failure with preserved ejection fraction (HFPEF). Morbidity and mortality of HFPEF are similar to HFREF; however, medications proven effective in HFREF have not been found to be effective in HFPEF. At present, there are no approved treatments to reduce mortality in HFPEF. In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in subjects with HFPEF, and are not approved therapies for HFPEF.

### Prophetic Example 6

The following prophetic example serves to provide a combination therapy for subjects with HFPEF, which includes a FAFD with a statin. To date there has been no prospective studies of statins in subjects with HFPEF. However, statins have pleotropic effects, in which they have been shown to be beneficial to non-HFPEF subjects beyond what was predicted based on their ability to reduce cholesterol, likely through anti-inflammatory pathways. By combining a statin with a FAFD, a synergistic effect to reduce the ROS associated with HFPEF is expected, which in turn will reduce stiffness in HFPEF and also reduce biomarkers such as IL-6, TNF-alpha, or NT-proBNP, and ultimately improve survival in HFPEF subjects. In one such example, a dose range between about 20 mg to about 5000 mg of the FAFD (I-1) is given to a subject with a statin dosage between 10 mg to 80 mg.

As used in the description and claims, the singular form “a”, “an” and “the” include both singular and plural references unless the context clearly dictates otherwise. For example, the term “prodrug” may include, and is contemplated to include, a plurality of prodrugs. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a method, substance, or composition.

As used herein, the term “comprising” or “comprises” is intended to mean that the methods and compositions include the recited elements, and may additionally include any other elements. “Consisting essentially of” shall mean that the methods and compositions include the recited elements and exclude other elements of essential significance to the combination for the stated purpose. Thus, a method or composition consisting essentially of the elements as defined herein would not exclude other materials, features, or steps that do not materially affect the basic and novel characteristic(s) of the claimed disclosure. “Consisting of” shall mean that the methods and compositions include the recited elements and exclude anything more than a trivial or inconsequential element or step. Embodiments defined by each of these transitional terms are within the scope of this disclosure.

The terms “optionally” as used herein means that a subsequently described event or circumstance may but need not occur, and that the description includes instances where the event or circumstance occurs and instances in which it does not.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating heart failure with preserved ejection fraction in a subject having heart failure with preserved ejection fraction, wherein treating comprises one or more of: reducing ventricular stiffness and improving ventricular relaxation in the subject, the method comprising administering to the subject in need thereof a therapeutically effective amount of a compound comprising one or more dialkyl fumarates of the formula: 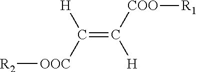
wherein R1 and R2, which may be the same or different, independently represent a linear, branched, or cyclic, saturated or unsaturated C1-20 alkyl radical which may be optionally substituted with a halogen, hydroxy, C1-4 alkoxy, nitro, or cyano; and optionally suitable carriers and excipients, and
wherein the compound is in the form of microtablets or micropellets.

2. The method of claim 1, wherein the one or more dialkylfumarates is dimethylfumarate.

3. The method of claim 1, wherein an amount of the one or more dialkylfumarates corresponds to 10 to 300 mg of fumaric acid.

4. The method of claim 1, wherein the compound is formulated into an oral preparation in which the microtablets or micropellets are in capsules or sachets.

5. The method of claim 1, wherein the microtablets or micropellets are provided with an enteric coating.

6. The method of claim 1, wherein the compound is formulated into an oral preparation in which the microtablets or micropellets are in capsules or sachets, and wherein the amount of dialkylfumarate corresponds to 10 to 300 mg of fumaric acid.

7. The method of claim 1, wherein the microtablets or micropellets are provided with an enteric coating and wherein the amount of dialkylfumarate corresponds to 10 to 300 mg of fumaric acid.

8. The method of claim 1, further comprising a preparation comprising the compound and one or more second agents selected from the group consisting of: a diuretic, an ACE-inhibitor, a beta-blocker, an angiotensin receptor blocker, isosorbide dinitrate, hydralazine, an angiotensin receptor-neprilysin inhibitor, an aldosterone antagonist, a PDE5 inhibitor, a statin, an angiotensin receptor blocker, a neprilysin inhibitor, an aldosterone inhibitor, an antitumor necrosis factor-alpha therapy, and a combination thereof.

9. A method of reducing mortality in a subject having heart failure with preserved ejection fraction, the method comprising administering to the subject a therapeutically effective amount of a compound comprising one or more dialkyl fumarates of the formula: 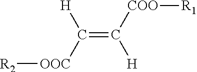
wherein R1 and R2, which may be the same or different, independently represent a linear, branched, or cyclic, saturated or unsaturated C1-20 alkyl radical which may be optionally substituted with a halogen, hydroxy, C1-4 alkoxy, nitro, or cyano; and optionally suitable carriers and excipients.

10. The method of claim 9, wherein the one or more dialkylfumarates is dimethylfumarate.

11. The method of claim 9, wherein an amount of the dialkylfumarates corresponds to 10 to 300 mg of fumaric acid.

12. A method of treating heart failure with preserved ejection fraction in a subject having heart failure with preserved ejection fraction, wherein treating comprises increasing maximal myocardial oxygen consumption in the subject, the method comprising administering to the subject in need thereof a therapeutically effective amount of a compound comprising one or more dialkyl fumarates of the formula: 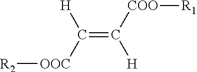
wherein R1 and R2, which may be the same or different, independently represent a linear, branched, or cyclic, saturated or unsaturated C1-20 alkyl radical which may be optionally substituted with a halogen, hydroxy, C1-4 alkoxy, nitro, or cyano; and optionally suitable carriers and excipients.

13. The method of claim 12, wherein the one or more dialkylfumarates is dimethylfumarate.

14. The method of claim 12, wherein an amount of the dialkylfumarates corresponds to 10 to 300 mg of fumaric acid.

15. The method of claim 12, wherein the maximal myocardial oxygen consumption is increased to greater than 14 mL/g/min.
